(19) United States
(12) Patent Application Publication
(10) Pub. No.: US 2011/0003331 A1
(43) Pub. Date: Jan. 6, 2011

(54) Method for enhanced production of biofuels and other chemicals using biological organisms

(76) Inventor: Thomas Clayton Pavia, Mojave, CA (US)

Correspondence Address: THOMAS CLAYTON PAVIA, P.O. BOX 998, MOJAVE, CA, 93502, US

(21) Appl. No.: 12/799,640

(22) Filed: Apr. 27, 2010

Related U.S. Application Data
(60) Provisional application No. 61/215,091, filed on May 2, 2009.

(51) Int. Cl. C12P 1/00; C12M 1/00

(52) U.S. Cl. 435/41; 435/289.1

(57) ABSTRACT

The invention of this disclosure, also known as the method of this disclosure, provides the means of improved Chemical Product production from chemical production systems that use biological organisms to produce the desired chemicals. This improved production is by means of subjecting chemical producing organisms to vibration waves or to an electrical voltage potential. The waves or voltage accomplish one of two things or both: they will improve the extraction of the chemicals from the bodies and cells of organisms, or they will increase the rate at which the organisms synthesize chemicals. To illustrate how one of many possible chemical production systems can incorporate this method, a specific configuration for a continuous chemical production system called the Oil Production System 90 is presented in this disclosure for the production of an example chemical: Vegetable Oil that is used as an ingredient in making biofuels.

### PRIORITY

This application claims the benefit and priority of U.S. Provisional Patent Application, Titled ‘Method for Enhanced Production of Biofuels and Other Chemicals Using Biological Organisms’, Ser. No. 61/215,091 filed 2 May 2009 under 35 U.S.C. 119(e).

### CROSS-REFERENCE TO RELATED NON-PROVISIONAL APPLICATIONS

NONE.

### FEDERALLY SPONSORED RESEARCH

NO

### SEQUENCE LISTING

NONE.

### FIELD

This disclosure relates generally to the production of chemicals from biological organisms, especially micro-organisms. More specifically chemical production by organisms that are subjected to vibration or electric voltage or both.

### KEY TERMS

In this disclosure the terms chemical product production system 60 or chemical production system 60 or Chemical Product 130 production system 60 are equivalent and are defined as a system of hardware that utilizes biological organisms 50 for the production of at least one chemical called the Chemical Product 130. The term Chemical Product 130 refers to one or more chemicals produced by the biological organisms 50 in the chemical production system. The term organisms 50 and organism 50 are equivalent to each other and can refer to one or more organisms but most often refers to a plurality of organisms such as in many micro-organisms. The terms body 40, cell 40, or cellular body 40 of single-cell organisms are equivalent to each other; the body of multi-cell organisms or a cluster of single-cell organisms being made up of cells 40 or cellular bodies 40.

### BACKGROUND

Biofuels are combustible or flammable fuels or ingredients of fuels that are produced by living organisms or quasi-living organisms, either unicellular (single-cell) or multi-cellular. Other useful chemicals can be produced by biological organisms as well. Using currently available technologies these useful chemicals are removed from the organisms usually with a process that destroys the organisms such as squeezing them in a press or subjecting them to vibrations that are sufficient to shatter the cellular bodies of the organisms thus destroying or killing the organisms. This destroying of organisms limits the rate at which the Chemical Product 130 can be removed from a given volume, weight, or number of organisms in the chemical product production system 60.

### SUMMARY OF METHOD

This invention was generated as an improvement for chemical product production systems using biological organisms to produce a chemical called the Chemical Product 130. This invention accomplishes this improvement in at least one of two possible ways:

A.) This invention makes possible the removal of the Chemical Product 130 from the organisms without destroying or killing at least a portion of the organisms that are producing the Chemical Product 130. In most applications of this invention most or all of the chemical-producing organisms are not destroyed or killed when the Chemical Product 130 is removed from them, or

B.) this invention makes possible, depending on the specific organism, an increase the rate of Chemical Product 130 production by the organisms in a Chemical Product 130 production system (as in pounds per hour of Chemical Product 130 produced by a given quantity of organisms). This is an increase in rate of production above and beyond the rate of Chemical Product 130 produced without the use of this invention, or both of these improvements are accomplished for a given chemical production system.

The above improvements provided by this invention are accomplished by subjecting chemical-producing organisms to a vibration or electrical voltage environment.

The chemicals that can be produced by this invention include but are not limited to fuels or so called ‘biofuels’ in addition to other chemicals. To illustrate and explain this invention, one of many chemical production systems that can use and incorporate this method will be described for the production of a Chemical Product 130 generically known as Vegetable Oil 116, a main ingredient for making biodiesel and other biofuels. One of many possible configurations of this invention for producing Vegetable Oil 116 is a chemical product production system 60 called the Oil Production System 90 of FIG. 1. After the Oil Production System 90 and its components are described, then various options and variations to this invention will be described.

The organisms that can be used with this invention include but are not limited to diatoms, protozoa, bacteria, algae, mold, micro-algae, seaweed, pond scum, macro algae, viruses, fungi, and other organisms. This invention can be used with either a plurality of organisms or singular organisms. In this disclosure the term ‘organisms’ usually, but not always, refers to a plurality of ‘micro-organisms’ such as in a cluster or colony of micro-organisms. Micro-organisms are defined as organisms that are small enough to require a microscope to discern or visually see individual organisms. Micro-organisms are sometimes designated as those organisms smaller than approximately 40 microns (or 0.001575 inches).

Such Biofuels or ingredients of fuels or other chemicals that can be produced by living organisms using this invention include but are not limited to oils, hydrocarbons, vegetable oil, lipids, fats, biodiesel, ethanol, methanol, alcohols, hydrogen, butane, methane, and other chemicals and compounds. Some other chemicals that can be produced by living organisms using this invention also include pharmaceuticals, food, food additives, food supplements, drugs of various kinds, and still other chemicals not listed here. The production of biofuels and biologically produced chemicals can be enhanced and increased for a given quantity of biological material (organisms) by subjecting the organisms to either sonic waves, or ultrasonic waves, or acoustic waves, or vibrations, or to an electrical Voltage Potential 280 (also known simply as voltage), or to any combination of any of these. Any sonic waves, ultrasonic waves, acoustic waves, or vibrations will be known in this disclosure as Vibration Waves 184.

Ultrasonic waves have a frequency greater than 20 kHz and are not considered as audible by the human ear. Sonic waves have a frequency lower than 20 kHz and are considered as audible (hearable) waves to the human ear and are simply known as ‘sound waves’. Historically acoustic waves have also been defined as ‘audible sound waves’ or simply as ‘sound waves’ as well, but in recent times acoustic waves are considered to include a wide range (frequency range) of waves traveling through a medium including sonic waves and ultrasonic waves. For the purposes of this disclosure ‘acoustic waves’ shall also include waves at very low and ultra low frequencies and very high and ultra high frequencies, in other words any wave traveling through a medium, despite the wave's frequency. In addition, for this disclosure the terms ‘waves’ and ‘vibrations’ are considered as equivalent to each other and therefore interchangeable with each other.

### BRIEF DESCRIPTION OF INVENTION

Brief descriptions of some of the benefits of this method are as follows:

Enhanced Separation of the Chemical Product from the Organisms: This invention provides a means of separating a Chemical Product 130 from the body/cell of a biological organism or biological organisms without destroying a portion or all of the individual organisms that have produced the chemical and contain it within or on their bodies or cells. This separation is accomplished by stressing, or stimulating, or agitating, or shaking the organisms with waves or vibrations or subjecting the organisms to an electrical voltage potential to induce the organisms to release the Chemical Product 130 without destroying the majority or at least a portion of the organisms thus effected. Once the organisms have released their Chemical Product 130 the organisms continue to make new Chemical Product 130 that will be continuously or repeatedly abstracted with the means described in this method. For example, some species of algae consist of up to 50-80% by weight Vegetable Oil 116 content (the primary ingredient of biodiesel fuel). When the algae are subjected to sonic, or ultrasonic, or acoustic waves its oil content can decrease to about 30%. When the vibration or waves are terminated the algae can again continue its Vegetable Oil 116 production until its weight is about 50% (or another value) Vegetable Oil 116 again which can once again be abstracted with this method, with this process proceeding on a continuous or repeated basis. Thus this method provides a means of abstracting a Chemical Product 130 from organisms on a continuous or repeated basis without destroying at least a portion or all of the organisms, which then can be reused to make new Chemical Product 130. As an example of this process the algae species Botryococcus braunii can contain up to 61% or more of its weight as Vegetable Oil 116. When this algae species is stressed, stimulated, or agitated, it can hold only approximately 31% of its weight as Vegetable Oil 116. Thus if it is stressed, stimulated, or agitated by vibrations or waves, it will release a significant portion of its Vegetable Oil 116 content and will then be ready to make new Vegetable Oil 116 after the vibrations or waves are terminated. Vegetable Oil 116 is a generic term for a substance produced by algae and other microorganisms. It can be a fatty substance or very much like the vegetable oil that is sold in a grocery store or be similar to other substances as well. The terms stressing, stimulating, agitating, or vibrating the organisms or subjecting the organisms to a voltage will be now referred to as ‘stimulating the organisms’. The terms Stimulus 290, Stimulant 290, or Stimuli 290 are equivalent to either Vibration Waves 184 or Voltage Potential 280 or both. Because some organisms are single-cell organisms the body of such organisms is the same as the cell of these organisms.

Separation of Chemical Product from the Growth Medium: Organisms often need some kind of substance or media in which to survive, grow, and reproduce. This so-called Growth Medium 132 can be any substance in which the organisms can survive, but in the case of algae, the Growth Medium 132 is often water and can be fresh water, brackish water, salt water or other types of water depending on the exact species of algae. Once the Chemical Product 130 has been removed from the organisms, this invention provides a means of removing the Chemical Product 130 from the mix that is comprised of both the Growth Medium 132 and the organisms (the mix of the two being called the Culture Solution 112), details of which are described below as part of the Oil Production System 90 of FIG. 1.

Increased Chemical Product Growth/Production Rate by the Organisms: While some organisms are stressed into releasing their Chemical Product 130 when subjected to sonic or ultrasonic waves or acoustic waves or vibrations or to a voltage potential, some organisms increase their growth rate when subjected to such stimuli, and thus can increase the growth rate of their body mass and/or of a Chemical Product 130 that they are producing as part of their growth process. For example, some organisms will increase their growth rate if subjected to an electrical voltage, the applied voltage depending on the voltage sensitivity of the specific organism. After such organisms that have been exposed to vibration or voltage stimuli have reached their harvest growth size, their Chemical Product 130 can be removed by the methods of this invention or by other means. Or, the growth rate of some organisms can be increased with this method with the intent of using a portion of or the entire bodies of the organisms as a product. For example, replacing the burning of coal in a power plant with the burning of a portion or all of the body of micro-algae or macro-algae (i.e. the Vegetable Oil 116 is not removed but the algae is burned whole) that has undergone enhanced growth with the use of this invention.

### FIGURES

FIG. 1 shows the layout of the Oil Production System 90 for the production of Vegetable Oil 116 by Algae 114 (micro-organisms or micro-algae).

FIG. 2 shows an end cross-section view of the Falling Film Bioreactor 92 and Solar Canopy 190.

FIG. 3 shows the details of the Oil Removal Tank 120.

FIG. 4 shows the details of the Oil Settling Tank 134.

FIG. 5 shows an alternative configuration of the Oil Production System 90 that utilizes Centrifugal Separators 224 to separate the Vegetable Oil 116 from the Culture Solution 112 (as opposed to using a Oil Settling Tank 134).

FIG. 6 shows the details of a Combined Oil Tank 192 that combines the functions of the Oil Removal Tank 120 and Oil Settling Tank 134 and utilizes rising Air Bubbles 158 to separate the Vegetable Oil 116 from the Culture Solution 112.

FIG. 7 shows a cross-section view of a Water Fall Bioreactor 94 with Floating Insulation 182.

FIG. 8 shows a Landfill Bioreactor 95 with Positive and Negative Electrodes 236, 238 producing electrical voltage potential across the Landfill 240 for enhancing Chemical Product 130 production (gaseous methane in this example) by organisms in the landfill.

FIG. 9 shows a Landfill Bioreactor 95 with Wave Generators 118 for enhancing Chemical Product 130 production (gaseous methane in this example) by organisms in the landfill.

FIG. 10 shows a cross-section of an overflow-type option for an Injection Manifold 103.

FIG. 11 shows an end cross-section view of the Solar Canopy 190 with Air Gap 180, Vents 260, and Air Gap Blower 210.

FIG. 12 shows a side cross-section view of a Solar Canopy 190 with a Canopy Blower 214 and Cryogenic Air Dryer 218.

Note that arrows in the tubing, pipe, channels, tanks, pools, gaps, or flow lines in the figures indicate direction of flow for Culture Solution 112, Vegetable Oil 116, a combination of fluids, Water 320, or Air 270 as helpful. Also note that figures are not to scale and show only details in the figures that are required to illustrate the scope of this disclosure.

### DETAILED DESCRIPTION OF THIS METHOD

The production of chemical products using this method may often require, but not limited to, three main components: a Bioreactor 100, a mechanism for Chemical Product 130 removal from the organisms, and a mechanism for Chemical Product 130 removal from the Growth Medium 132.

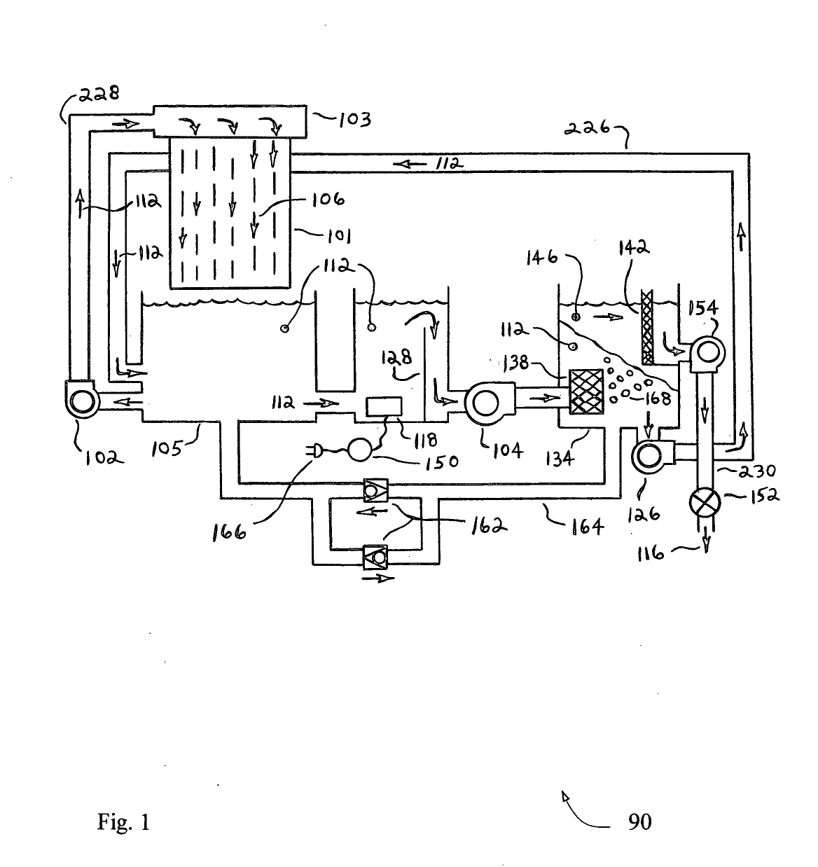

Bioreactor: a generic Bioreactor 100 is a mechanism in which biological organisms or microorganisms can grow and multiply. The bioreactor typically supplies the type of environment (i.e. nutrients, gases, Growth Medium 132, temperature range, ph range, and others) that the organisms need to survive and thrive. It also provides photo-dependent organisms the artificial light or natural sunlight they require to survive and thrive. For this purposes of this disclosure the term bioreactors can refer to either a fabricated device (as will be described below), or an ocean, sea, lake, river, stream, swamp, marsh, waste pond or waste stream, sewage pond, sewage stream, landfill, or other artificial or natural system that organisms can survive in. In the Oil Production System 90 shown in FIG. 1 the bioreactor is a Falling Film Bioreactor 92 that is comprised of a Pool 105, Falling Film Ramps 101, Injection Manifold 103, Culture Solution 112, Culture Pump #1 102, piping connecting the Pool 105 to the Injection Manifold 103, and any controls or equipment for maintaining nutrient level, temperature, ph level, flowrates, etc. The bioreactor of FIG. 1 is only one of many types of bioreactors or bioreactor configurations that can be used with this invention.

Mechanism for Chemical Product Removal From Organisms: As described above this is a mechanism providing a means of subjecting the organisms to sonic, or ultrasonic, or acoustic waves, or vibrations, or electric voltage potential at the correct level, amplitude, frequency, power level, and duration to induce the organisms to release all or a portion of the Chemical Product 130 that the organisms contain within their cells or bodies or is attached to the organisms. In the Oil Production System 90 of FIG. 1 this mechanism is referred to as the Oil Removal Tank 120 since in the Oil Production System 90 of FIG. 1 the main Chemical Product 130 is Vegetable Oil 116.

Mechanism for Chemical Product Removal from Growth Medium: Organisms or microorganisms are often suspended in a Growth Medium 132 that allows them to grow and multiply. For example, such as algae growing in water, water being the Growth Medium 132. After the sonic, or ultrasonic, or acoustic waves, or vibrations, or electric voltage potential separates the Chemical Product 130 from the organisms, a mechanism must then be used to separate the Chemical Product 130 from the Growth Medium 132. In the Oil Production System 90 of FIG. 1 it is a mechanism that is specifically designed to remove the Vegetable Oil 116 from the Culture Solution 112 and will be called the Oil Settling Tank 134. Note that the Culture Solution 112 is usually not a true ‘solution’ in that the organisms are not usually dissolved in the Growth Medium 132 but are suspended, swimming in, or otherwise living in the Growth Medium 132.

### Apparatus of Method

### Description of Oil Production System Components

The Oil Production System 90 of FIG. 1 is only one specific type of chemical producing configuration that can use this invention. Other variations, types, options, configurations, and Chemical Products 130 of a chemical production system consistent with this method are possible. The Oil Production System 90 of FIG. 1 is only one specific configuration for producing one specific chemical for the purposes of illustrating this invention. Other variations are possible where helpful.

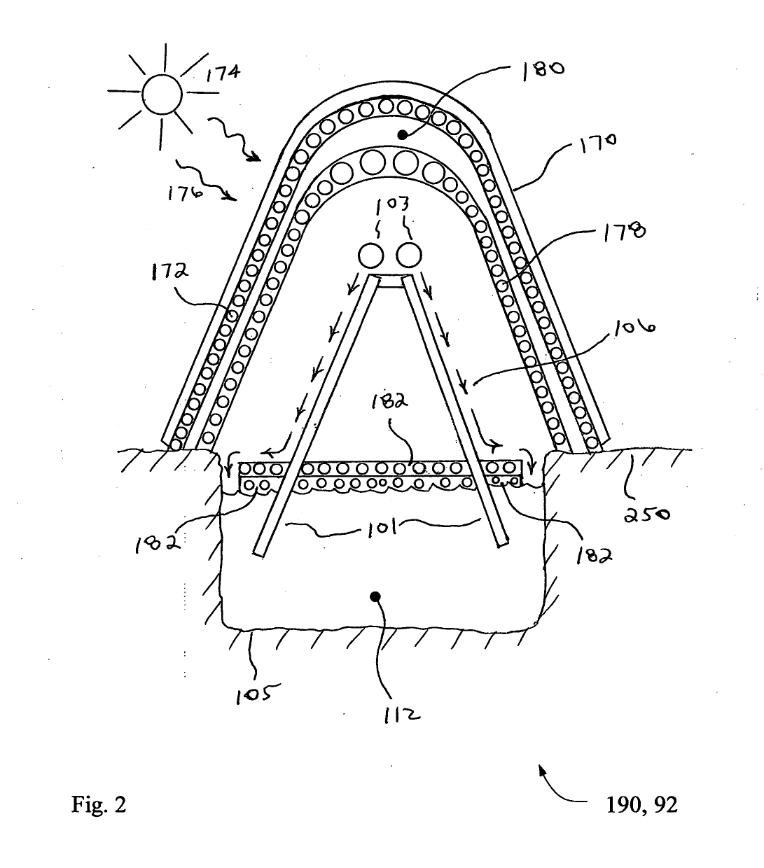

The Oil Production System 90 of FIG. 1 is a Vegetable Oil 116 (the main ingredient in biodiesel fuel) production unit utilizing a Falling Film Bioreactor 92 (FIGS. 1 and 2). The organisms in this case are micro-algae 114 (sometimes generically referred to as ‘pond-scum’ or simply ‘algae’) growing in a water medium (called the Growth Medium 132). The mixture of the two will be henceforth called the Culture Solution 112, the Culture Solution 112 being the combination of the Algae 114 and the water. The water can either be freshwater, saltwater, brackish water, or water with other ingredients. The water for the Oil Production System 90 is brackish water. In this system the Culture Solution 112 is contained in a Pool 105. A Culture Pump #1 102 (see FIG. 1) pumps the Culture Solution 112 to the top of the Falling Film Ramp 101 (simply known as the ‘Ramp’) where the Culture Solution 112 is distributed across the top of the Ramp 101 through an Injection Manifold 103 which can be of any quantity, size, or shape but is sometimes, but not limited to, a perforated tube or pipe. The Culture Solution 112 then exits the Injection Manifold 103 through a series of holes or slots (or equivalent) to flow across the face of the Ramp 101 as layer of fluid (called a Falling Film 106) that falls down the Ramp 101 back into the Pool 105. The Injection Manifold 103 can be a single manifold or multiple manifolds. In FIG. 2 it is shown as two manifolds. The Culture Solution 112 falling down the Ramp 101 accomplishes three things for the Algae 114:

a.) It exposes the Algae 114 to atmospheric or artificially provided gases it requires to thrive such as carbon dioxide (CO2) and other gases.
b.) It exposes the Algae 114 to sunlight or artificial light that is needed for the photosynthesis required to produce the resulting Vegetable Oil 116.
c.) It allows the Culture Solution 112 to release excess gases such as oxygen or other gases thus preventing excess gas saturation of the Culture Solution 112.

Algae 114 typically can have a composition of up to and exceeding 50% Vegetable Oil 116. The Vegetable Oil 116 is abstracted and then is converted to biodiesel or other biofuels. Thus the Culture Solution 112 is constantly re-circulated by Culture Pump #1 in a closed loop from the Pool 105 to the top of the Ramp 101 and then back down the Ramp 101 again to the Pool 105.

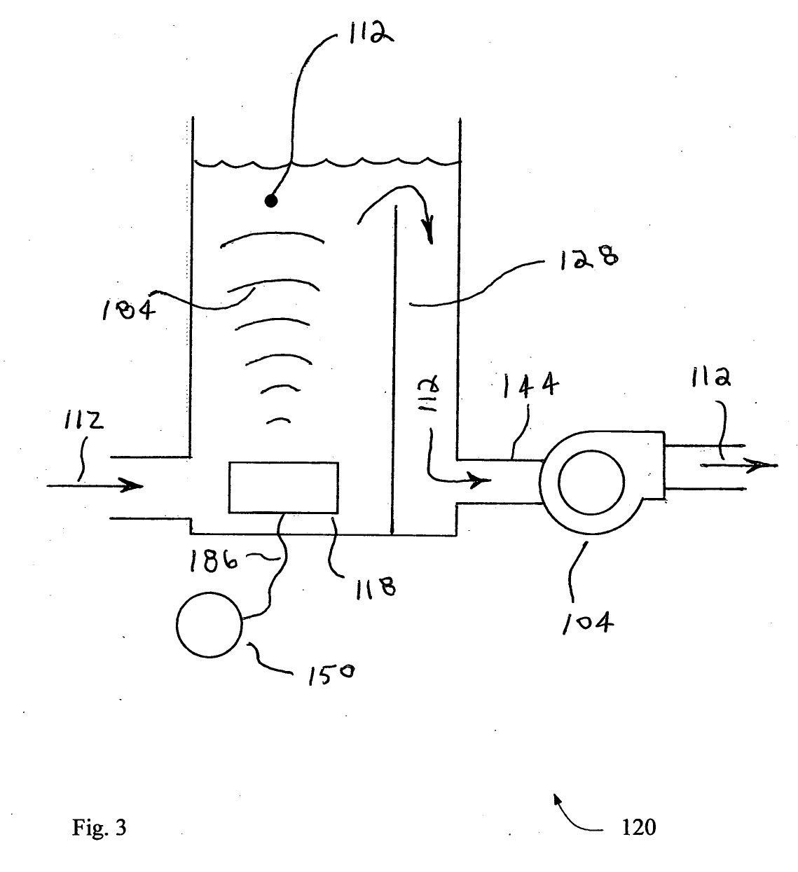

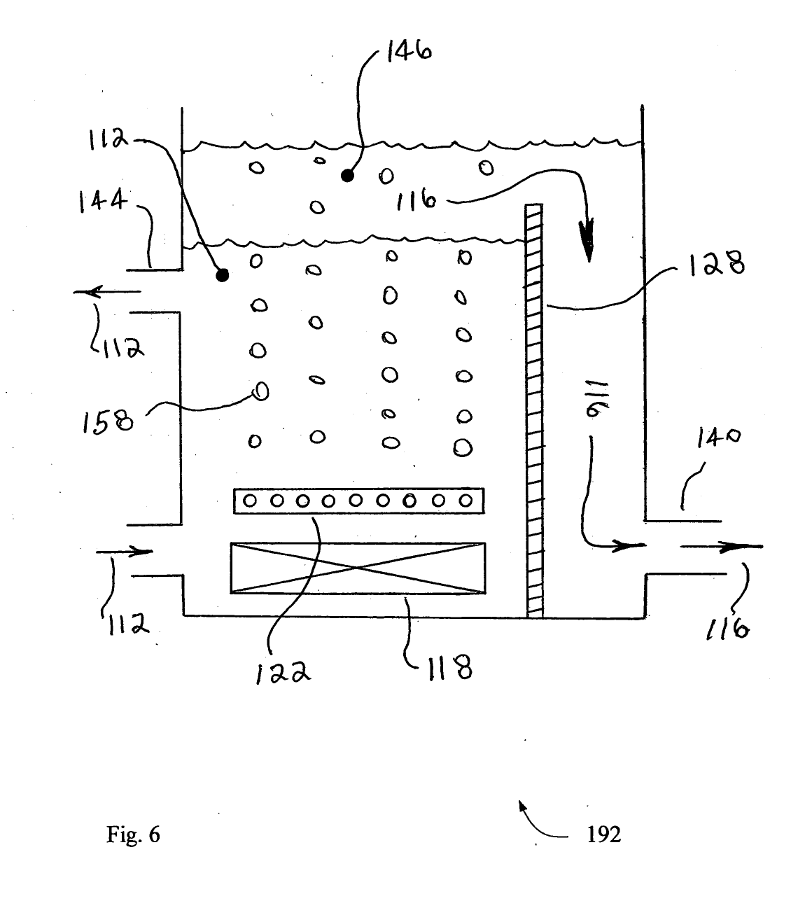

The Oil Removal Tank 120 as shown in FIGS. 1 and 3 performs the function of removing the Vegetable Oil 116 from the algae cells while the Oil Settling Tank 134 removes the Vegetable Oil 116 from the Culture Solution 112. It is an option to combine these two functions in one device or tank or vessel but is shown in this Oil Production System 90 as two separate tanks for clarity purposes. See FIG. 6 for one of many possible configurations of a tank or vessel that combines the functions of the Oil Removal Tank 120 and the Oil Settling Tank 134 by utilizing rising gas bubbles to separate the Vegetable Oil 116 from the Culture Solution 112. Such a tank will be called a Combined Oil Tank 192.

In summary, as shown in FIG. 1, the Culture Solution 112 flows from the bioreactor's Pool 105 to the Oil Removal Tank 120 where the Algae 114 cells in the Culture Solution 112 are subjected to Vibration Waves 184 produced by a Wave Generator 118. The Vibration Waves 184 cause the Algae 114 to release a portion or all of its Vegetable Oil 116 into the Culture Solution 112. The mix of the Culture Solution 112 and the now released Vegetable Oil 116 flow out of the Oil Removal Tank 120 and into Culture Pump #2 104. Culture Pump #2 104 pumps the Culture Solution (with the released Vegetable Oil 116) through the Coalescing Pack 138 where the finely dispersed Vegetable Oil 116 coalesces into larger oil droplets that float to the top surface of the Culture Solution 112 in the Oil Settling Tank 134. The Vegetable Oil 116 accumulating at the top of the Oil Settling Tank 134 in an Oil Layer 146 then flows out the Oil Outlet 140 into the Oil Pump 154 that pumps the Vegetable Oil 116 to the appropriate holding or transfer tank, device, vessel, or location. The Culture Solution 112 comprising of water and Algae 114 flows out through the Culture Solution Outlet 144 to Culture Pump #3 126 that transfers the Culture Solution 112 back to the bioreactor's Pool 105. Of course, Culture Pump #1 108 continuously pumps Culture Solution 112 to the top of the Ramp 101 where the Culture Solution 112 flows down the Ramp 101 as a Falling Film 106 back into the Pool 105 where Culture Pump #1 pumps the Culture Solution 112 back to the top of the Ramp 101 again on a continuous basis.

What is unique about the Falling Film Bioreactor 92 is the following:

Full Light Penetration: Typically Sunlight 176 can only penetrate a mix of Algae 114 and water up to a depth of 1″ to 12″ depending on the light intensity and algae concentration. The depth of the Falling Film 106 on the bioreactor Ramp(s) 101 will most often be, but not limited to, less than 1″ in thickness. This ensures that most or all of the Algae 114 (or any other photosynthetic organisms) will get the greatest possible exposure to Sunlight 176 or artificial light when flowing down the Ramp 101; light that is necessary for photosynthesizing processes that generate Vegetable Oil 116.

No Optical Surfaces to Foul: In many bioreactors that contain organisms that are engaged in photosynthesis, the organisms (in the Culture Solution 112) obtain their required light by the Culture Solution 112 flowing through a clear container such as a transparent plastic tube or glass container. In such Bioreactors 100 the organisms are in contact with and growing on the transparent surfaces that are transmitting light to the organisms. Because of this the organisms growing on the transparent surfaces are rendering those surfaces less and less transparent to light as the organisms grow (i.e a process called ‘fouling’). This gradually reduces the light exposure to most of the organisms in the Culture Solution 112 and inhibits the photosynthesis that the organisms generate in order to produce Chemical Product 130. In a Falling Film Bioreactor 92 there is no fouling of a transparent light-transmitting surface because there is no surface with organisms growing on it that stands between the light and the organisms.

Falling Film Evaporation Cools/Heats Local Environment: Since the Falling Film 106 flowing down the Ramps 101 is open to the local atmosphere it can evaporate some of its water on hot days and thus cool the Culture Solution 112 and the local environment (such as the interior of a Solar Canopy 190). This allows the Solar Canopy 190 and the Falling Film Bioreactor 92 to work together to produce an acceptable temperature environment for the organisms (Algae 114 in the Oil Production System 90). Likewise on cool days, since the Falling Film 106 has direct exposure to the local atmosphere it can transfer some of its heat to the atmosphere. This would be most effective when the Falling Film 106 is transferring solar heat that it absorbed during the day to the interior space of the Solar Canopy 190 at night. A warmer Solar Canopy 190 interior would help lessen heat loss from the Culture Solution 112 on a cold night.

Low Cost Bioreactor: Falling Film Bioreactors 92 can be made of but not limited to low cost materials such as common chicken wire and plastic film and still be effective in supporting the growth of organisms.

Optimal Light Exposure: The angle of the falling film Ramp 101 (or Ramps), with respect to the sun or available light source, can be set for optimal exposure to the available light source such as setting the angle so that the Ramp is directly facing the light source or at the best average angle for optimal overall solar exposure.

Good Falling Film Stability: Since the Falling Film 106 has a Ramp 101 under it, the Ramp 101 well help ensure that the Falling Film 106 does not break up (due to turbulence) over a longer distance than if the film were simply falling unsupported in open air. The longer the distance the Falling Film 106 can flow without significant turbulence, the more light will penetrate the Falling Film 106, and the more effective the light will be in driving photosynthesis in the organisms.

Increases Land Usage Effectiveness: With a common type of bioreactor called a Raceway Pond (a pond shaped like a racetrack) the effective land area that is exposed to sunlight is the top surface of the Raceway Pond. For a Falling Film Bioreactor 92 the surface area exposed to sunlight can be multiplied many times by the presence of Ramps 101. For example, if a Pool 105 is four feet on a side then its solar exposure area is 4 feet times 4 feet or a total of 16 square feet. However if the same Pool 105 has two falling film Ramps 101 mounted in it and each Ramp 101 is 4 feet by 12 feet then the total solar exposure area of the Falling Film Bioreactor 100 would be a total of 112 square feet (includes the Pool 105 area) or 7 times greater that the solar exposure area of the Pool 105 alone. Thus the Falling Film Bioreactor 92 offers the ability to greatly increase Chemical Product 130 yield for a given plot of land by increasing the surface area exposed to solar energy and thus increasing the photosynthesis activity (i.e of Chemical Product 130 production rate) of the organisms in question.

### Apparatus of Method

### Oil Removal Tank

In the Oil Production System 90 the design and function of the Oil Removal Tank 120 is as follows:

The Culture Solution 112 is routed to the Oil Removal Tank 120 at some flowrate.

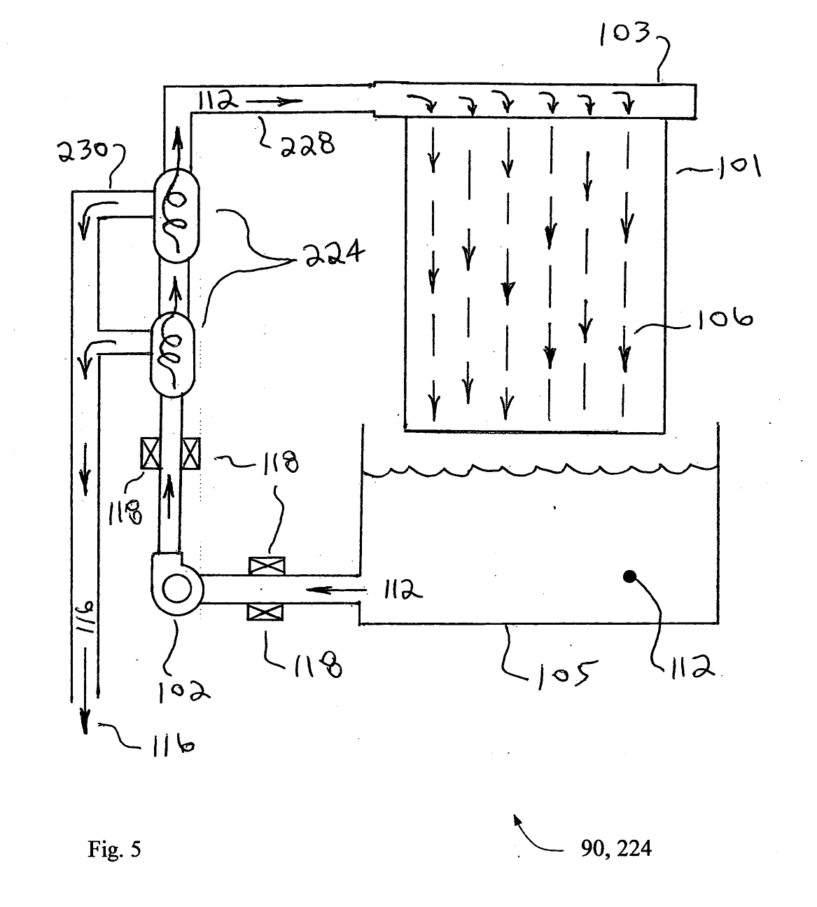

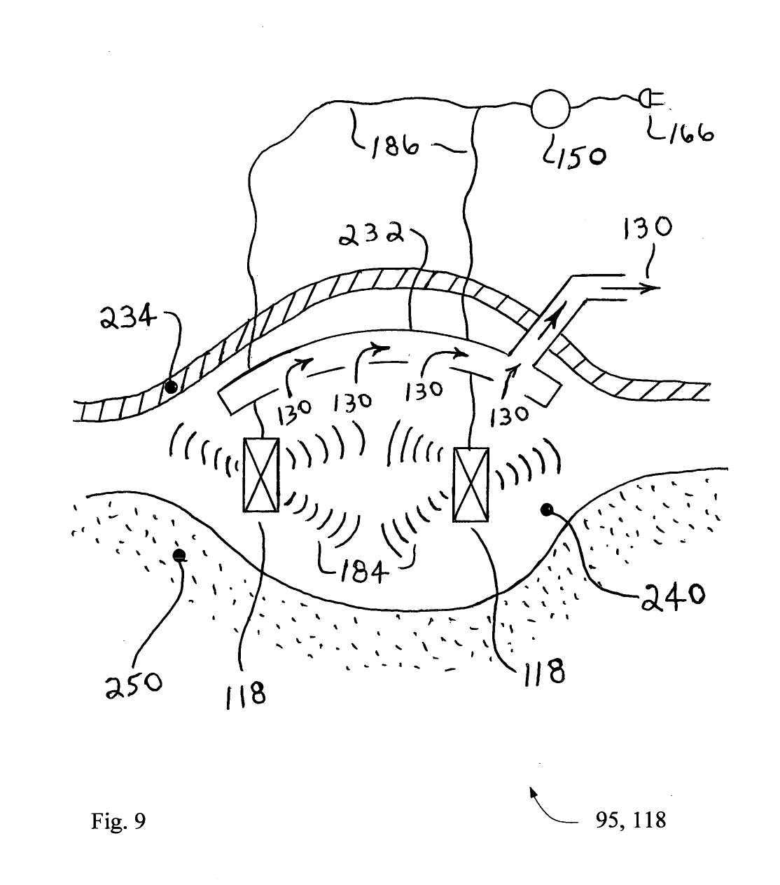

In or attached to the Oil Removal Tank 120 is an ultrasonic wave generator (called a Wave Generator 118 in this disclosure) that is set to a specific amplitude, power level, and frequency or is controlled to a range or variation of power levels, amplitudes, and frequencies. The Wave Generator 118 can be powered by any means so long as it generates the correct frequency or frequencies at the correct amplitude or amplitudes for the appropriate duration for making the Algae 114 (or other organism) release all or a portion of their Vegetable Oil 116 (i.e. or other Chemical Product 130). In the Oil Production System 90 the Wave Generator 118 is operated in a continuous mode at a fixed frequency and amplitude. The Wave Generator 118 can be mounted on the inside or outside surface of the Oil Removal Tank 120, or simply placed inside the Oil Removal Tank 120, or placed in contact with the Oil Removal Tank's 120 outer surface. The point is that the exact location of the Wave Generator 118 is not critical so long as it (or they) can properly stimulate the Culture Solution 112 to the correct frequency, amplitude, power level, and duration or any plurality of these. Although the Oil Production System 90 utilizes only one Wave Generator 118 it is an option that any number of Wave Generators 118 can be used anywhere in the Oil Production System 90 where helpful and can be either controlled in unison or independently of each other or have no control at all. In literature the Wave Generators 118 are often referred to as acoustic horns; horns; sonic or ultrasonic generators; sonic, acoustic, or ultrasonic wave generators; vibrators; sonic, ultrasonic, or acoustic transducers; or by other names. If more than one Wave Generator 118 is used in any one Oil Production System 90 such as exemplified in FIGS. 5 and 9, then it is an option that the Wave Generators 118 can be turned on and off in an alternating manner, or sequenced, or phased, or have their frequency or amplitude adjusted with respect to each other such that the Vibration Waves 184 from the multiple Wave Generators 118 do not cancel each other out or detrimentally impact each other.

When the Algae 114 in the Culture Solution 112 are subjected to the ultrasonic waves generated by the Wave Generator 118 the Algae 114 will be stimulated to release all or a portion of the Vegetable Oil 116 contained within them into the water (or other media). This method of oil removal from the algae is unique and unlike other methods of oil removal using sonic, ultrasonic, or acoustic waves, or vibrations. Current technology methods rely on the waves/vibrations to break apart the algae cells and release the Vegetable Oil 116 thus destroying the organisms. The method of oil removal of this invention does not bombard the algae cells with waves/vibrations capable of destroying the organisms, but just enough to cause the organisms (Algae 114 in the Oil Production System 90) to release their Vegetable Oil 116 in a way that allows all or most or a portion of the organisms to continue living and producing more Vegetable Oil 116. The duration of exposing the organisms to the vibrations/waves can be anywhere from seconds to minutes to hours depending on the type of organism that is being affected by the vibrations, the power level of the Wave Generator 118, the Wave Generators 118 output characteristics such as amplitude and frequency, and the amount of Chemical Product 130 to be removed from the organisms.

The Oil Removal Tank 120 can be either a closed or open tank but is shown as an open tank in the Oil Production System 90. It is sized such that the residence time of the Algae 114 as it flows through the tank is great enough to ensure that the Algae's contact time with the vibrations from the Wave Generator 118 is long enough to separate a quantity of Vegetable Oil 116 from the Algae cells.

The Controller/Power Supply 150 connected to the Wave Generator 118 in the Oil Removal Tank 120 can either be a combination of a controller and power supply or these two functions can be separate units or, if preferred, the Wave Generator(s) 118 can be run without a controller. The Controller/Power Supply 150 can be of any type that will do the job including a controller that is electronic, electrical, mechanical, fluidic, or others. The Electric Plug 166 shown in FIGS. 1, and 9 or any other figure is an example only and is strictly optional depending on how the Controller/Power Supply 150 is powered and hooked up.

### Apparatus of Method

### Oil Settling Tank

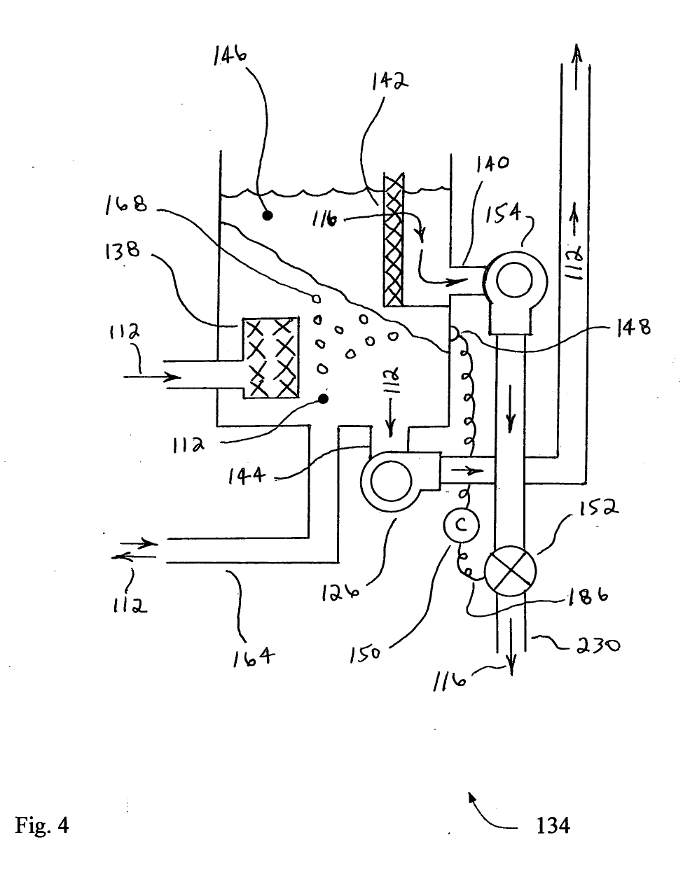

The Oil Settling Tank 134 (FIGS. 1 and 4) function and design is as follows:

The Oil Settling Tank 134 has traits that are unique to this disclosure. An oil/water separator is needed because the microscopic Algae 114 would be producing Vegetable Oil 116 that is very finely suspended in the Culture Solution 112. The Oil Settling Tank 134 operates by the oil-water mix (or oil-Culture Solution 112 mix) flowing through a coalescing media pack (called the Coalescing Pack 138) where the finely suspended oil droplets impact onto the porous media material in the Coalescing Pack 138, accumulates, and forms oil droplets that are large enough to float to the top surface of the water/Culture Solution 112 in a short period of time (i.e. a few seconds is typical but sometimes shorter or longer).

The media material of the Coalescing Pack 138 must have pores or flow passages through it that are large enough for the Algae 114 to flow through the Coalescing Pack 138 without plugging the Coalescing Pack 138. After reaching the top surface of the water/Culture Solution 112, the oil is drawn off through its own exit port called the Oil Outlet 140. The coalescing media material is a porous element with a large surface to volume ratio. The coalescing media can be made of stainless steel, fibers, glass, plastic, or other materials. The traits of the oil-water separator tank (called the Oil Settling Tank 134 in this disclosure) that are unique to this disclosure is that the Oil Settling Tank 134 of the Oil Production System 90 can separate not only oil from water but can separate Vegetable Oil 116 from a mix of water and living organisms (such as micro-algae), the mix being the Culture Solution 112. And, this will be accomplished in a mechanically simple manner with a low expenditure of energy.

The first unique attribute of the Oil Settling Tank 134 is that the Coalescing Pack 138 is treated or fabricated with a repelling material (called the Bio-repellant 136) that will prevent algae (or other organisms) from bonding to, anchoring itself, or growing on the media in the Coalescing Pack 138. Bio-repellant 136 is a material substance that the Algae 114 (or other organisms) are repelled by and thus avoid. The Bio-repellant 136 is often copper or copper compounds, but other materials can be used as well. Bio-repellant 136 can also include other metals, metal compounds, organic materials, inorganic materials, or any material that repels or is detrimental to Algae 114 or the specific organism requiring repelling. The Bio-repellant 136 material can be incorporated into the Coalescing Pack 138 media as either a plating, coating, embedded material, paint, a material loosely or rigidly incorporated into or mixed into the Coalescing Pack 138 base material during manufacture, or by other methods.

The function of the Bio-repellant 136 is to prevent the organisms from fouling or clogging the Coalescing Pack 138. Algae 114 or other organisms would normally attempt to avoid flowing through a treated Coalescing Pack 138 but in the Oil Settling Tank 134 the Culture Solution 112 is forced through the Coalescing Pack 138 by Culture Pump # 2 104. Once through the Coalescing Pack 138 the now coarser/larger droplets of Vegetable Oil 116 will float more quickly to the Culture Solution's 112 top surface (oils that are less dense than water float on water) towards the Oil Outlet 140 than oil that is finely suspended in water.

Another attribute that makes the Oil Settling Tank 134 unique is a BioFilter 142 that prevents the Algae 114 from being drawn out of the tank through the Oil Outlet 140, but keeps all or portion of the Algae 114 flowing out of the Oil Settling Tank 134 (along with the water) through the Culture Solution Outlet 144. The BioFilter 142 is a porous media element that contains, is fabricated with, or is treated with Bio-repellant 136 as is the Coalescing Pack 138. What this does is causes the Algae 114 to propel itself away from the BioFilter 142 in order to prevent contacting the Bio-repellant 136. This is possible because Algae 114 and other organisms usually have a means of propulsion that allow themselves to avoid locations that are undesirable for continued growth and prosperity. Thus their tendency would be to stay with the water and not pass through the BioFilter 142 with the Vegetable Oil 116.

Once through the BioFilter 142, it is an option that the Vegetable Oil 116 can continue on through any additional number of Oil Settling Tanks 134, Coalescing Packs 138, and/or BioFilters 142 to filter out remaining water and Algae 114, with the Vegetable Oil 116 being pumped into an appropriate Vegetable Oil 116 storage, shipping containment, or usage. As with the Coalescing Pack 138, the BioFilter 142 must have passage sizes that are large enough to allow the Algae 114 to pass through it without clogging the BioFilter 142. The reason that the Algae 114 passes through the Coalescing Pack 138 is that Culture Pump #2 104 forces it through the Coalescing Pack 138. However, a portion or all of the Algae 114 will not flow through the BioFilter 142 because the fluid velocity flowing through the BioFilter 142 is set at a low enough velocity such that the Algae 114 can propel themselves away from the BioFilter 142 before they flow through it.

One means (not the only one) of setting this fluid velocity through the BioFilter 142 is by proper sizing of the BioFilter 142 dimensions. Like the Coalescing Pack 138, the BioFilter 142 can be constructed of any material that has numerous flow passages through it. Such materials include sintered metals (sintered copper or sintered brass as an example), open-cell plastic foam, fiber or wire wound elements using any type of fiber or wire, cloth elements using any type of cloth or fabric, beads or other shapes made of any material of acceptable compatibility with the Growth Medium 132 and organisms. This would include shapes made of glass, plastic, or metal and others, plates with holes, formed shapes of any material, complex metal, glass, or plastic shapes, and other materials and shapes.

It is shown in FIG. 4 that an Oil Layer 146 will form around the tank's Oil Outlet 140. To keep this Oil Layer 146 at the appropriate thickness around the Oil Outlet 140 a Sensor 148 is placed such that it can detect the thickness of the Oil Layer 146. The Sensor 148 can be an optical sensor, a capacitance sensor, a pressure sensor, or any other type of sensor capable of sensing or determining Oil Layer 146 thickness, orientation, or geometry. When it senses the Oil Layer 146 thickness, the Sensor 148 conveys its output to a Controller/Power Supply 150 that controls a Throttle Valve 152 that adjust the flowrate of the Vegetable Oil 116 out of the tank's Oil Outlet 140. This in turn adjusts the location of the Oil Layer 146 relative to the BioFilter 142 element(s). This control method is strictly an option and the system can be run without such a control method if it is calibrated and/or adjusted properly, such as control using orifices, tube sizes, or flow adjusting valves, on/off valves, manual valves, and other methods. In addition, the Throttle Valve 152 can be any type of valve that can be controlled by the Controller/Power Supply 150. The Controller/Power Supply 150 can be a separate unit or be built into the Sensor 148 or the Throttle Valve 152 itself.

Note in FIG. 4 that the BioFilter 142 element is oriented vertically. This is because if the BioFilter 142 element is oriented horizontally the organisms rising with the Oil Droplets 168 would be more likely to pass through the horizontal BioFilter 142 porous element. This is strictly an option since the BioFilter 142 can be of any shape, orientation, thickness, or size that is preferred or helpful, with as many porous surfaces as preferred or helpful. It is also possible to have no Biofilter 142. In some cases the organisms involved finds a pure or near pure oil environment to be intolerable and therefore will steer clear of the Oil Layer 146 on their own accord. In this case the Vegetable Oil 116 will flow out the Oil Outlet 140, without all or a portion of the organisms, due to pressure or overflow. It simply depends on what kind of organism is being utilized. To separate out the Vegetable Oil 116 any number of BioFilter 142 elements or Oil Settling Tanks 134 can be used in series or in parallel. If Algae 114 becomes trapped between two BioFilter 142 porous elements then an option would be to push the Algae 114 back into the Culture Solution 112 with a Vegetable Oil 116 purge or other type of purge.

### Apparatus Of Method

### Alternative Combined Oil Removal/Settling Tank

An alternative tank for oil removal and settling is shown in FIG. 6. This tank combines the functions of the Oil Removal Tank 120 and Oil Settling Tank 134 into one tank, the Combined Oil Tank 192. This Combined Oil Tank 192 removes the Vegetable Oil 116 from the Algae 114 with a Wave Generator 118 as described above and then coalesces the Vegetable Oil 116 into larger droplets using gas bubbles rising in the Culture Solution 112 as opposed to using a Coalescing Pack 138. A Coalescing Pack 138 could be used as preferred or helpful in the Combined Oil Tank 192 but is not used in the specific configuration shown in FIG. 6. In this specific configuration, in or mounted to the Combined Oil Tank 192 is an Air Manifold 122. The Air Manifold 122 injects bubbles of air, or carbon dioxide, or an appropriate gas or mixture of gases (generically called Air Bubbles 158) into the Combined Oil Tank 192. As the Air Bubbles 158 rise to the top surface of the Culture Solution 112 in the Combined Oil Tank 192 they create a ‘lifting current’ that carries the Vegetable Oil 116 towards the top surface of the Culture Solution 112 (i.e. the mix of Algae 114 and water). This occurs because the Vegetable Oil 116 is typically less dense than water or algae and has a tendency to float upward, thus separating itself from the water by floating on top of the Culture Solution 112. In the Oil Production System 90 the Air Bubbles 158 generated are very small, more like a gentle fog than large spherical air bubbles. If the action of the Air Bubbles 158 is too strong, it will cause a remixing of the Vegetable Oil 116 and Culture Solution 112 as opposed to assisting in their separation. However, it is an option to make the Air Bubbles 158 of any size that is deemed to be helpful.

When the Vegetable Oil 116 floats to the top surface of the Culture Solution 112 in the Combined Oil Tank 192 it forms an Oil Layer 146 at the top surface of the Culture Solution 112. As seen in FIG. 6 an Oil Outlet 140 receives the Vegetable Oil 116 overflow off of the top surface of the Culture Solution 112. The Vegetable Oil 116 is then routed to its ultimate destination, or if any water still remains in the Vegetable Oil 116, it is routed to any of the separators described in this invention or any available separator capable of separating water from Vegetable Oil 116. Note that in the configuration of FIG. 6 no BioFilter 142 is used since this particular configuration is for use with organisms in the Culture Solution 112 that are not attracted to a pure oil environment and would thus propel themselves away form the Oil Layer 146. The residence time of any one unit of Culture Solution 112 in the Combined Oil Tank 192 must be sufficient duration to separate the Vegetable Oil 116 from the Algae 114 and for the separated Vegetable Oil 116 to float to the Oil Layer 146 at the top of the tank.

As shown in FIG. 6 there is a Baffle 128 inside the Combined Oil Tank 192 to keep the Culture Solution 112 flowing evenly throughout the Combined Oil Tank 192 on its way to the Culture Solution Outlet 144 and Culture Pump #3 104. The Culture Pump #3 104 then pumps the Culture Solution 112 back to the Pool 105 or to whatever type of Bioreactor 100 is being used. Since the Oil Layer 146 is not an ideal environment for algae to grow as compared to water it will be mostly free of Algae 114 for certain species of Algae 114. Any Algae 114 remaining in the Vegetable Oil 116 as it is pumped from the Combined Oil Tank 192 can be removed with standard industrial means, additional Biofilters 142, or other means presented in this method.

### Apparatus Description

### Optional Solar Canopy

The Solar Canopy 190 as shown in FIG. 2 is optional but is very advantageous when used with a Bioreactor 100. This is because it greatly reduces the water loss due to evaporation, helps in preventing undesirable algae species and other organisms from contaminating the Culture Solution 112, and allows the Algae 114 (or other photosynthetic organism) to gather Sunlight 176 or artificial light while providing more temperature control of the organisms' environment (i.e. Algae 114 in the Oil Production System 90). Other variations, options, and alternatives to the Solar Canopy 190 are possible.

The Solar Canopy 190 of FIG. 2 is comprised of the Inner Insulation 178, Outer Insulation 172, Outside Covering 170, Air Gap 180, and other components discussed below. The Solar Canopy 190 of this invention is designed to maintain the appropriate environment for the organisms while being as low cost as possible. The reason for this is that to produce Vegetable Oil 116 or other chemical product in a large-scale production the Bioreactor 100 and Solar Canopy 190 must cover many acres and even many square miles. Both the Bioreactor 100 and Solar Canopy 190 can be used in very small sizes but for large-scale Vegetable Oil 116 or other product production they must cover a large area, so the amount of materials and labor used in their construction must be minimized.

A cross-section of the width of the Solar Canopy 190 and Falling Film Bioreactor 92 is shown in FIG. 2. The Solar Canopy 190 and the bioreactor in it can be any size or shape but for the Solar Canopy 190 of the Oil Production System 90 is shaped like an inverted ‘U’, longer than it is wide, with half-domed ends for added streamlining and wind resistance. Since Algae 114 typically (but not always) require an environment with a temperature between 65 degF and 95 degF in order to thrive, the Solar Canopy 190 is designed to operate in conjunction with the bioreactor in order to create an environment inside the Solar Canopy 190 that is warm enough in the winter and cool enough in the summer to allow the organisms to survive, reproduce, grow, and thrive. The basic means of accomplishing this is with transparent or semi-transparent insulation for the Solar Canopy 190 that is used in conjunction with an Air Gap 180 between the layers of Transparent Insulation 196. The function of the Transparent Insulation 196 and Air Gap 180 is to help remove excess solar heating from the Solar Canopy 190 in the summer and add and retain solar heating as required in the winter, all while allowing Sunlight 176 to penetrate the Solar Canopy 190 in order to drive photosynthesis in the Algae 114. More on this concept will follow.

The bioreactor of the Oil Production System 90 is a Falling Film Bioreactor 92 as shown in FIGS. 1 and 2. The Falling Film Bioreactor 92 of FIG. 2 has a Pool 105 that is dug into the ground, but other Pool 105 configurations are possible. The Pool 105 can be any size, depth, type (such as a Raceway Pond), or shape but for the Oil Production System 90 the Pool 105 will be rectangular. The Pool 105 can be used without any kind of Liner 198 (as in a plastic film liner) or it can have a Liner 198 (not shown in figures) made of any material or thickness that is acceptably compatible with the Culture Solution 112. This may include Liners 198 that are made of but not limited to plastic film, fiberglass, composites, plastic, metal, concrete, and others. For the Oil Production System 90 the Pool 105 Liner 198 is a polyethylene plastic film liner. For improved leak resistance the Liner 198 of the Oil Production System 90 is made of two layers of 3-mil plastic film (i.e. each 0.003″ thick) that are laminated together with an adhesive such as Silicone or other adhesives.

The Falling Film Bioreactor 92 shown in FIG. 2 is an ‘A-frame’ bioreactor with two opposing Ramps 101. A Falling Film Bioreactor 92 can have any number of Ramps 101, be of any height, size, or shape, or made of any material acceptably compatible with the Culture Solution 112. In keeping with the desire for lowest cost, the Falling Film Bioreactor 92 of the Oil Production System 90 can have the structure of its ‘A-frame’ constructed of, but not limited to, PVC plastic pipe and fittings that are glued and tie-wrapped together. On each of the PVC pipe Ramps 101 will be mounted a Mesh Wire Mat 200. Over the Mesh Wire Mats 200 will be mounted a polyethylene liner, the Ramp Liner 204, of the same construction as the Pool Liner 198. The purpose of the A-frame 202 is to support the Mesh Wire Mat 200. The purpose of the Mesh Wire Mat 200 is to support the Ramp Liner 204, and, of course, the Falling Film 106 of Culture Solution 112 that flows down and spreads out over the Ramp Liner 204.

The Ramp(s) 101 (includes A-frames and other Ramp structure) can be made of any material or technique acceptably compatible with the Culture Solution 112 including sheets of metal, plastic/fiber composite, treated or protected wood, and plastic film or sheet, and others materials. In the Oil Production System 90 the ramp Mesh Wire Mat 200 can be stainless steel chicken wire or stainless steel hardware cloth, plastic chicken wire or plastic hardware cloth, coated metal chicken wire or hardware cloth, fabric cloth, fibers strands, or other types of materials. The only requirement is that the Mesh Wire Mat 200 supports the Ramp Liner 204 when the Falling Film 106 is flowing on it.

As seen in FIG. 2 there are two layers of Floating Insulation 182 floating on the surface of the Culture Solution 112 in the Pool 105. In actuality the Floating Insulation 182 can be one or more layers and used in any location on or around the pool so long as it accomplishes its role of insulating the Culture Solution 112 to the preferred level. Other types of insulation can be used throughout the Oil Production System 90 as helpful.

The purpose of the Floating Insulation 182 is to keep the Culture Solution 112 at relatively constant temperature and to reduce the daily average inside temperature of the Solar Canopy 190 during the summer and to increase the average daily temperature inside the Solar Canopy 190 during the winter months. In other words the job of the Floating Insulation 182 is to help eliminate temperature extremes in the Solar Canopy 190 and in the Culture Solution 112 as will be discussed in the paragraphs to follow. The Floating Insulation 182 is shown in FIG. 2 in three locations: between the two Ramps 101 and to the side of each Ramp 101 for a total of three locations. However, the Floating Insulation 182 can be used in as many or as few locations as preferred or helpful. For example, it can be used only between the two Ramps 101 or not used at all. The Floating Insulation 182 can be made of any material of any thickness that is acceptably compatible with the Culture Solution 112 and has the appropriate insulation properties. This includes but is not limited to Bubble Pak, foam, floating ping-pong balls, and other materials and objects. The Floating Insulation 182 can either float on the Culture Solution 112, be above the Culture Solution 112, or be mounted in the most helpful location using any means to do so.

The Solar Canopy 190 for the Oil Production System 90 is constructed and operated as follows:

A.) Skeletal Structure: The main structural element of the Solar Canopy 190 is a wire-frame Skeletal Structure 208 (not shown in figures). The Skeletal Structure 208 establishes the basic shape of the Solar Canopy 190 and allows it to resist wind, rain, and snow pack loads. The Skeletal Structure 208 can be made of rods, wire, tubing, piping, struts of any shape, or other structural members. It can be made of any material or held together by any means so long as it can remain intact under the design loads. A Solar Canopy 190 can be of any size from small to large, but a typical range is 1/10 acre to several acres and larger; from the size of a family camping tent to a large circus tent and larger.

B.) Transparent Insulation (with Air Gap): For thermal control of the Solar Canopy 190 interior the main element is the Transparent Insulation 196. The Transparent Insulation 196 of the Oil Production System 90 is formed of the Inner Insulation 178, Outer Insulation 172, Air Gap 180, and Outer Covering 170. The Inner and Outer Insulation 178, 172 have heat-insulating properties but are transparent or semi-transparent to allow Sunlight 176 or artificial light into the Solar Canopy 190 to stimulate photosynthesis in the Algae 114. For the Oil Production System 90 common plastic bubble-pack (as is used in packaging) is used for the Inner and Outer Insulation layers 178, 172. The Outside Covering 170 is a transparent or semi-transparent layer that is made of woven fabric, plastic film, plastic sheet, glass, or other material. For the Oil Production System 90 the Outside Covering 170 will be a transparent or semi-transparent fabric that protects the insulation layers from mechanical damage from wind-borne particles, rain, birds, and rodents. It also will protect the insulation layers from damage due to ultraviolet light. In the Solar Canopy 190 design, to improve mechanical resistance to wind and snow, a mesh wire matting (made of any material) is placed on the outer surface of the Outside Covering 170, the inside surface of the Outer Insulation 172, the outside surface of the Inner Insulation 178, and the inside surface of the Inner Insulation 178. In the Oil Production System 90 the mesh wire matting (not shown in figures) can be secured to the Skeletal Structure 208 using tie-wire, or tie-wraps, clamps, or other means.

C.) Solar Canopy Thermal Control: The temperature control of the interior of the Solar Canopy 190 for the Oil Production System 90 depends upon the Falling Film Bioreactor 92 and the Solar Canopy 190 working together to control the Solar Canopy's interior environment. A possible but non-limiting scenario of thermal/temperature control is described below:

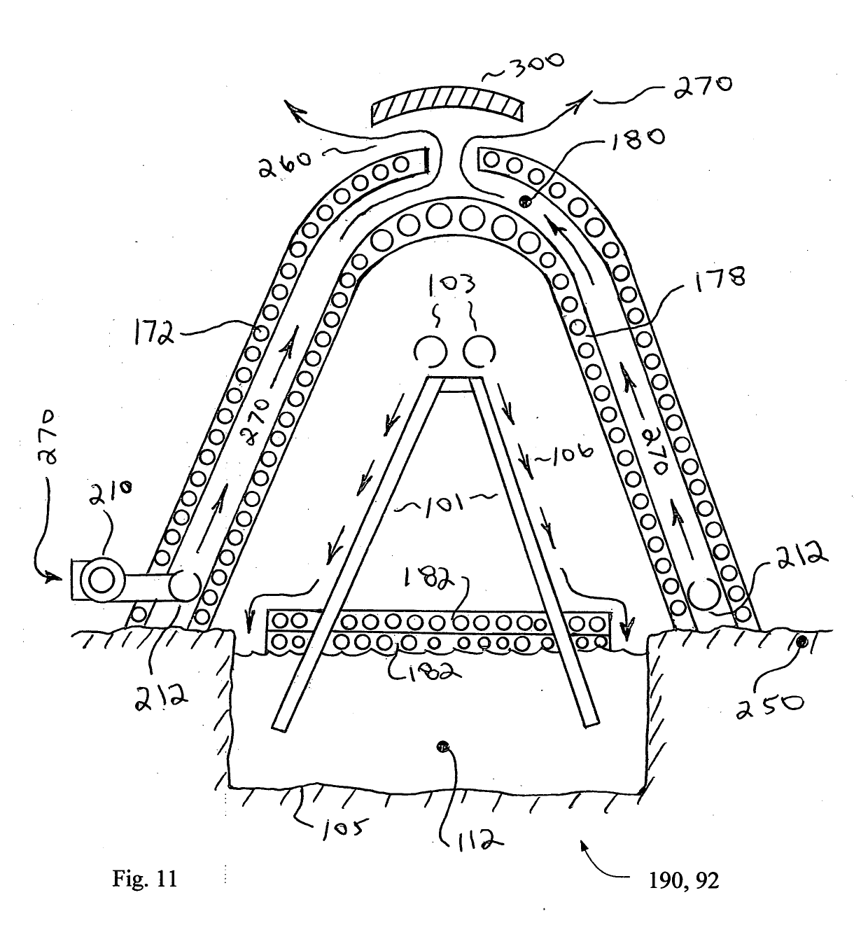

D.) Hot Days: On hot days an Air Gap Blower 210 will force Air 270 upward through the Air Gap 180 and out Vents 260 near or at the top or at other locations on the Solar Canopy 190. The Solar Canopy 190 can have any number, size, or configuration of Vents 260 as helpful. To distribute the Air 270 evenly around the perimeter of the Solar Canopy 190 the Air Gap Blower 210 will be blowing into a Purge Manifold 212 (FIG. 11) that is mounted in the Air Gap 180 around the base (i.e. bottom) of the Solar Canopy 190. The Purge Manifold 212 is a tube or pipe or other manifold that has holes, slits, or other openings through it that are sized such that the Air 270 from the Air Gap Blower 210 is distributed around the Solar Canopy 190 as required to maintain proper thermal control. This happens because much of the solar infrared heat that has gotten through the Outer Insulation 172 will heat the air in the Air Gap 180.

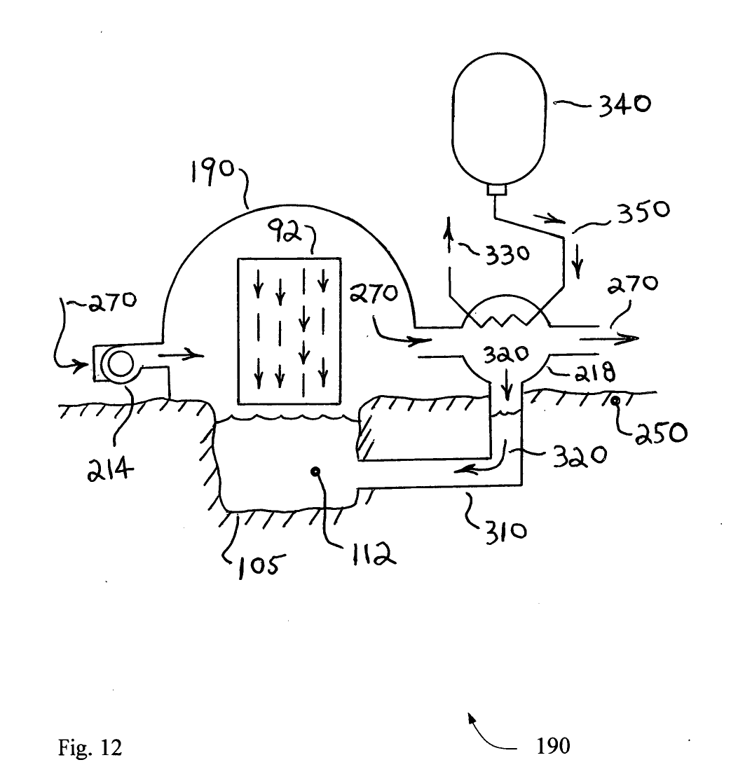

When the Air Gap 180 is purged by the Air Gap Blower 210 then much of that solar infrared heat is blown out the Vents 260 (as heated air) near the top of the Solar Canopy 190 and thus cannot be used to excessively heat the interior environment of the Solar Canopy 190. Whatever solar heat does get inside the Solar Canopy 190 will heat the canopy's internal environment. Thusly, undesirable heating on a hot day and will be counteracted in two ways: First, a Canopy Blower 214 (FIG. 12) will start and begin blowing Air 270 into the interior of the Solar Canopy 190 and out a vent, thus blowing heated Air 270 out of the Solar Canopy 190. Second, Culture Pump #1 102 is on and water from the re-circulating Culture Solution 112 of the Falling Film Bioreactor 92 will partially evaporate in the warm air. This will absorb some of the heat from the air inside the Solar Canopy 190 that is then blown out of the Solar Canopy 190 by the Canopy Blower 214. The combination of purging the Air Gap 180, blowing out the interior of the Solar Canopy 190 with the Canopy Blower 214, and water evaporation from the bioreactor will reduce the air temperature in the Solar Canopy 190 on a hot day to a value that is the same or lower than the outside ambient air temperature. These steps will also help prevent any overheating of the Culture Solution 112 and Algae 114. Any evaporated water that is blown out of the Solar Canopy 190 can be recondensed and rerouted to the Pools 105. The recondensing is accomplished by an industrial or custom Air Dehumidifier 216 or by a Cryogenic Air Dryer 218 or both. A Cryogenic Air Dryer 218 is a heat exchanger that recondenses wet air by cooling the air with evaporated or liquid cryogenic liquids such as liquid nitrogen or by semi-cryogenics such as carbon dioxide or other cryogens or semicryogens. After the water is recondensed it is routed back to the Pools 105 by a Water Return Pump 220 or by gravity.

E.) Hot Nights: On hot nights the Air Gap Blower 210 is off because there is no additional or excess solar heat, that needs to be removed, coming into the Air Gap 180. The Culture Pump #1 102 and the Canopy Blower 214 are on. This scenario will have the Culture Solution 112 continue re-circulating on the Ramps 101 which causes continued water evaporation which in turn cools the Culture Solution 112 and the interior of the Solar Canopy 190. The Canopy Blower 214 is on in order to blow out the freshly evaporated water that is then recondensed by the Air Dehumidifier 216 or Cryogenic Air Drier 218 or both. After recondensing the water is returned to the Pool(s) 105.

F.) Cold Days: On cold days the Culture Solution 112 has to absorb as much solar energy as possible. To this end Culture Pump #1 102 is on in order to re-circulate the Culture Solution 112. In addition, the Air Gap and Canopy Blowers 210, 214 are off to minimize any loss of heat from the interior of the Solar Canopy 190.

G.) Cold Nights: On cold nights Culture Pump #1 102 is off to allow the Floating Insulation 182 retain the heat in the Culture Solution 112. Likewise, the Air Gap and Canopy Blowers 210, 214 are off to minimize any loss of heat from the interior of the Solar Canopy 190.

Note that the above blower and pump sequences are optional and can be varied depending on the need to control temperatures of the Culture Solution 112 and the interior of the Solar Canopy 190. Any of the Culture Pumps or other pumps in the system can be activated depending on when the operator wishes to separate Vegetable Oil 116 from the Algae 114 and Culture Solution 112 or when helpful to control temperature. In a dry environment the average temperature of the interior of the Solar Canopy 190 and Culture Solution 112 will be approximately the average temperature resulting from solar heating, night heat loss, and cooling from the evaporation of water from the Culture Solution 112. In a humid environment water evaporation will be less significant thus the average temperature of the interior of the Solar Canopy 190 and Culture Solution 112 will be approximately the average of day and night air temperature with little or no water evaporation. The Air Gap 180 can have within it any structure or material for directing and diverting air flow as helpful.

### Some Method Options

Some, but not all, of the options for this invention include:

Bioreactor Type: The bioreactor or bioreactors used for chemical production using this method can be of any bioreactor type so long as it supports the biological needs of the living organisms producing the Chemical Product 130 that this invention enhances the removal of (from the organisms) or growth of. Such bioreactors can be of, but not limited to, the type of bioreactors that support photosynthesizing organisms (organisms that require artificial light or natural sunlight) such as Falling Film Bioreactors, tubular bioreactors, airlift bioreactors, so-called ‘Raceway Ponds’, plastic film bioreactors, or other types of bioreactors. Raceway Ponds and other types of ponds are simply called ‘ponds’ in industry, but for the purposes of this invention these ponds and other such devices that support the growth or reproduction of biological organisms will be called ‘bioreactors’. Or the bioreactors used with this method can be a simple device such as a pot or a fish tank or any other type of device that supports biological organisms, either photosynthesizing organisms or non-photosynthesizing organisms. Typically bioreactors supply organisms with a growth medium, nutrients, temperature control, ph control, salinity control, necessary gases, and sometimes artificial or natural light, among the other necessities for survival of the organisms. Usually, but not always, Bioreactors 100 also facilitate the removal of undesirable gases and other wastes from the organisms' growth media. The bioreactors used with this invention can be single or multiple bioreactors. Other types of bioreactors can be either fabricated (i.e. man-made) or natural and can include but are not limited to sewage ponds, waste ponds, oceans, seas, rivers, streams, lakes, ponds, marshes, swamps, bogs, landfills, tanks, tubs, vessels, and containers of all kinds, pools, and others. Bioreactors can be either open to the air or closed.

Chemical Product/Organism Separation Mechanism: The Oil Removal Tank 120 as shown in FIGS. 1 and 3 is strictly one specific configuration of this method and other configurations can be used for the mechanism that separates the Chemical Product 130 from the organisms using this method. The only requirement for such a mechanism is that it will stimulate the organisms to the proper frequency, amplitude, power level, duration, or voltage. An oil removal mechanism that utilizes vibration contains or has mounted in or onto it a Wave Generator 118 or Wave Generators 118 that can appropriately stimulate the organisms. The Wave Generator 118 can be any mechanism that can stimulate the organisms to the appropriate vibration environment. The Wave Generator can be mounted on or in a tank, pool, pond, pipe, tube, or other fixture using any methods of mounting (even loosely mounting the Wave Generator(s) 118) so long as the organisms are subjected to the appropriate vibration environment for removing the Chemical Product 130 from the organisms. To this end the Wave Generator 118 can be run in a fixed and continuous mode, or in a variable mode where the Wave Generator 118 is pulsed on and off at any repeat rate or wave shape that is effective in removing Chemical Product 130 from the organisms, or the Wave Generator 118 vibration amplitude or power output can gradually or rapidly rise and fall at any effective repeat rate, or the Wave Generator 118 frequency or amplitude can be varied during the Chemical Product 130 separation process, or multiple Wave Generators 118 can be used in the same tank, pool, pond, pipe or tube with at least one Wave Generator 118 operating with one vibration regime and at least one Wave Generator 118 operating at other vibration regimes whether the regimes are fixed and continuous or variable.

Multiple Wave Generators 118 can be used in the same system so long as they are controlled such that the Vibration Waves 184 from the multiple Wave Generators 118 do not cancel each other out or detrimentally effect each other. To remove the Chemical Product 130 from any one organism, that organism can be subjected to the vibration environment for a duration of seconds or minutes or hours or other time durations depending on the specific organism's vibration sensitivity and the amount of Chemical Product 130 to be removed. In FIG. 1 the Oil Removal Tank 120 is shown as being separated from the main Pool 105 of Culture Solution 112. This only an option and does not have to be the case. For instance the Oil Removal Tank 120 can be located within the main Pool 105 itself or it can be the entirety or a portion of the Pool 105 with at least one Wave Generator 118 located in or around a portion of or the entirety of the Pool 105 or pond or bioreactor. Likewise the mechanism for oil removal (i.e. removal of the Chemical Product 130) can be a totally enclosed tank, pipe, pool, pond, vessel, or tube or open on one or more sides.

Chemical Product/Organisms/Growth Media Separation Mechanism: In addition to separating the Chemical Product 130 from the organisms, the Chemical Product 130 must also be separated from the medium (called the Growth Medium 132) in which the organisms are growing and/or suspended. Thus in any Chemical Product 130 production system once the Chemical Product 130 is separated from the organisms by means of this invention, the Chemical Product 130 must then be separated from the Culture Solution 112 with an appropriate mechanism. As an alternative or as an addition to the Oil Settling Tank 134 of this disclosure, the vibration and voltage method of this disclosure can be used with any custom or currently available or commercial mechanisms for separating fluids where helpful. Such separation methods include but are not limited to settling mechanisms, centrifugal separation, vacuum separation, filtering, static charging, cyclone separators, reverse osmosis, and others. As an example of such devices used with this method see FIG. 5 (discussed further below).

Pumps, Valves, Sensors, Controls, etc. Any number of pumps, valves, vents, ports, relief valves, blowers, power supplies, check valves, orifices, throttle valves, needle valves, manual valves, actuated valves of all kinds, sensors, controllers, and other items for control, safety, material handling, material transport, and other reasons can be used with this invention where helpful. These devices will be henceforth known as Control Devices 96. The valves, pumps, and controllers shown in FIG. 1 are for example purposes only. This invention can be used with any number of Control Devices 96 (even no Control Devices 96 at all) placed anywhere in the chemical production system where helpful. The pumps can be any type or number of pumps or pumping mechanisms (such as paddle wheels or airlift pumps) driven by any energy source so long as the pumps or pumping mechanisms can move a liquid Culture Solution 112 or Growth Medium 132 or other fluid requiring transport. The valves can also be any type or number of valve that is helpful. Also, the various components, pools, vessels, containers, flow passages, plumbing, canopies, or tanks in the invention system can contain any number of baffles, dividers, or flow diverters where preferred or helpful. Any number of pools, tanks, vessels, pipes, ponds, vessels, and other containers can be used with this invention when and where helpful.

Combined Functions: Any number of tanks, pools, bioreactors, Chemical Product 130 removal tanks (for example, the Oil Removal Tank 120), Chemical Product 130 settling tanks (for example, the Oil Settling Tank 134) can be used with this invention with any number of stages of Chemical Product 130 separation from the organisms, Growth Medium 132, or Culture Solution 112. The functions of various tanks, pools, vessels, pumps, plumbing components, and other components can be combined or integrated with each other where helpful. For example, the functions of the Oil Removal Tank 120 and the Oil Settling Tank 134 of the Oil Production System 90 can be incorporated into one tank with the Wave Generator(s) 118, Coalescing Pack 138, and BioFilter 142 existing in one tank. Such functions can also be combined with the Bioreactor 100 tank, vessel, or Pool 105 where helpful. A single tank could also settle out the Chemical Product 130 using any combination of air bubbles (i.e. gas bubbles), Coalescing Packs 138, and BioFilters 142. Coalescing Packs 138 and Biofilters 142 are optional and not necessary if other mechanisms are used to fulfill the same functions.

Current Technology Separation Methods: Currently available (i.e. current technology) material separation methods for removing the Chemical Product 130 from the Growth Medium and Culture Solution can be used with this invention when preferred. Such methods include but are not limited to settling mechanisms, centrifugal separation, vacuum separation, filtering, static charging, cyclone separators, reverse osmosis, and others. Other configurations are possible. In the specific configuration of FIG. 5 two current technology, commercial available Centrifugal Separators 224 are used in series with each other to remove the Vegetable Oil 116 from the Culture Solution 112. As shown in FIG. 5, the Chemical Product 130 is removed from the organisms by mounting Wave Generators 118 on the outside surface of the tube or pipe leading to the Centrifugal Separators 224 (as a specific configuration only, other configurations and mounting schemes are possible). These Wave Generators 118 transmit their vibrations through the pipe or tube and into the Culture Solution 112 that is flowing in the pipe or tube in order to separate the Chemical Product 130 from the organisms.

Component Size, Shape, Orientation, Material, Geometry, Thickness: Any component of the Chemical Product 130 production hardware or system (one of many configurations of which is the Oil Production System 90 of FIG. 1) can be any size, shape, number, orientation, geometry, material, thickness, or other feature as helpful. For example, tanks, vessels, containers, or pools can be of the open or closed type; can be separate or integrated with the Bioreactor 100 or each other; any number of inlets or outlets to tanks or components can be used as preferred or helpful; fluid inlets or outlets can be simple overflow, or pressurized, or other types of inlets or outlets, the flow of Culture Solution/Growth Medium 112/132 through the product production hardware can be made possible by any number of pumps or static head pressure or other pressure source or any combination of these; Coalescing Packs or BioFilters can be of any number, size, or orientation where helpful (such as mounted vertically or horizontally or other orientations).

Flow Passages: Culture Solution/Growth Medium flow passages connecting tanks, vessels, pools, or other components to each other can be pipes, tubing, channels, streams, canals, trenches, hoses, or any other passages as helpful and can be utilized as helpful; and any component in the Chemical Product 130 production hardware can be fabricated of any material of acceptable compatibility to allow production of the Chemical Product 130. Examples of fluid flow passages are the Solution Return Line 226, the Bioreactor Recirculation Line 228, and the Oil Discharge Line 230 shown in FIG. 1 and other figures.

Lighting, Nutrients, Gases, etc.: Components and substances for sustaining living organisms can be added as necessary to any component or hardware in this invention. These Life Sustaining Components 160 can include but not be limited to components that add to the system such things as lighting, nutrients, gases, temperature control, ph control, salinity control, Growth Medium 132 or Culture Solution 112 quality, flowrate control, and other components for sustaining the living organisms in a healthy and living state.

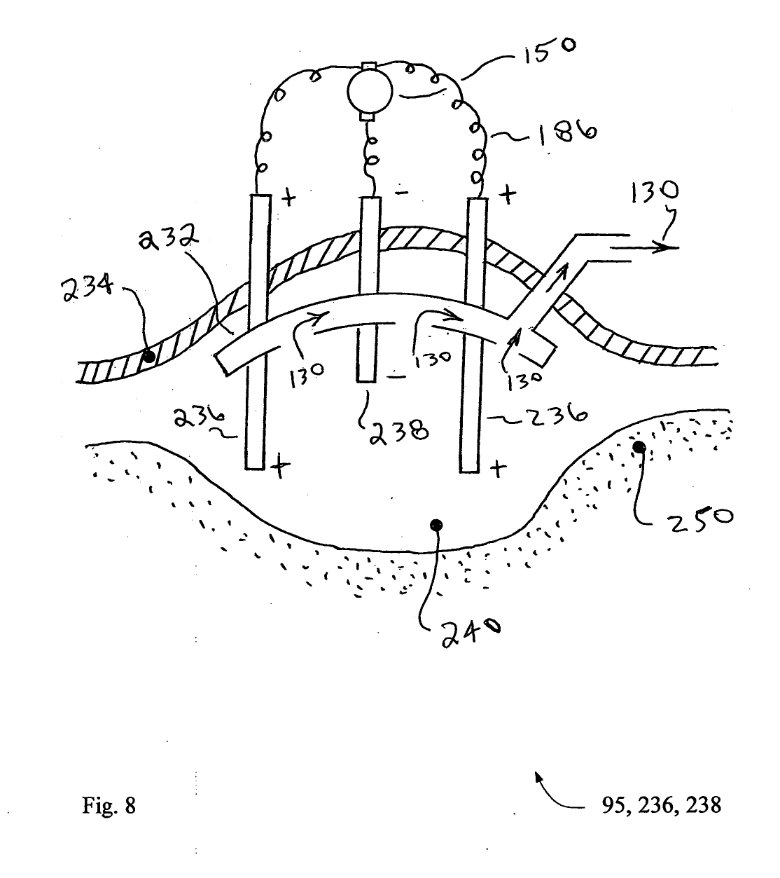

Landfills: As mentioned previously, a chemical production system utilizing this invention can utilize a landfill as a Bioreactor 100. Landfill Bioreactors 95 can be used in, but are not limited to, the production of useful gaseous fuels such as methane, ammonia, or hydrogen. Into the Landfill Bioreactor 95 are fed the necessary gases and liquids (for example, but not limited to air, nitrogen, and water) to produce a Chemical Product 130 such as methane as an example. The methane or other gas is collected in a manifold that is often but not always a series of pipes (in or on the landfill) with holes in the pipes after which the methane or other gas is then ducted to the appropriate receiving tank, use, or device. Usually, the methane is produced in the landfill by bacteria that is digesting the waste material contained within the landfill. To increase this production of Chemical Product 130 by the organisms in the landfill, the landfill can be either stimulated by Wave Generators 118 as in FIG. 9 or by a voltage as in FIG. 8. FIG. 8 shows Positive and Negative Electrodes 236, 238 for creating the voltage potential across the landfill. In FIG. 9 Wave Generators 118 are on top of or buried in the landfill to stimulate the organisms in the landfill to increase Chemical Product 130 production (in this case methane, but other products are possible). FIG. 8 shows a similar means of increasing the production of Chemical Product 130 but is accomplished by stimulating the organisms within the landfill by Positive and Negative Electrodes 236, 238 in or on top of the landfill; electrodes that provide an electric voltage (i.e. the electrodes are charged to a voltage potential) that can be either high or low voltage, in some cases ten volts or less. The voltage can be applied in either the direct current (DC) or alternating current (AC) manner, or both. In some cases the Ground 250 can act as one of the electrodes. Subsequently the Chemical Product 130 produced by the organisms in the landfill (usually but not always the useful organism in landfills is bacteria) is collected into the ‘holed’ pipes called Gas Collection Manifolds 232 in or on the landfill and is routed to an appropriate receiving tank, use, or device. In addition to using either vibration or electrical voltage to stimulate the production of Chemical Product 130 in a landfill, any combination of vibration or electrical voltage can be also used. The methods discussed above are also applicable to other types of bioreactors either man-made or natural such as waste ponds, sewage ponds, other types of ponds, Falling Film Bioreactors 92, Waterfall Bioreactors 94, tubular bioreactors, airlift bioreactors, and other types of bioreactors. FIGS. 8 and 9 also show an optional Topsoil Layer 234.

Continuous Processing, Batch Processing, or Semi-Continuous Processing:

A chemical product production system utilizing this invention can be run in either a mode of Continuous Processing or Batch Processing or Semi-Continuous Processing or any combination of these. A continuous processing method draws the Chemical Product 130 out of the chemical production system on a steady and continuous basis. Batch processing is where the Chemical Product 130 is allowed to accumulate in the chemical production system and then is drawn out of the chemical product production system as a lot or ‘batch’. A semi-continuous process is similar to the batch process with the exception that the ‘batches’ that are drawn out of the chemical product production system are more numerous and smaller in size than with the ‘batch process’.

Solar Canopy Options: Some, but not all, of the options to the Solar Canopy 190 include:

A.) The Inner and Outer Insulation layers 178, 172 can be any preferred or helpful thickness including zero thickness for either layer. The Air Gap 180 between the Inner and Outer Insulation 178, 172 can be any preferred or helpful thickness.

B.) The Inner and Outer Insulation layers 178, 172 can be made of any material that is at least partially transparent to light and is acceptable to the purpose of lighting and insulation. It is preferable but not necessary that the Inner and Outer Insulation layers 178, 172 and any other Solar Canopy 190 materials are resistant to flame and/or degradation by ultraviolet light.

C.) The Outside Covering 170 can be any transparent or semi-transparent cloth, plastic, plastic film, glass, composite, or other materials. Its primary job is to protect the underlying Transparent Insulation 196 layers from mechanical damage (such as from birds, wind impact damage, weather etc.) and/or from ultraviolet light damage, but it is an option to eliminate the Outside Covering 170 completely by incorporating its protective properties directly into the Transparent Insulation 196 layers. Other options for the Outside Covering 170 is that it can be a composite, plastic, glass, or plastic film that has variable tint properties. That is it can have properties such that it gets darker and more opaque with increasing light intensity so as to limit the temperature rise inside the Solar Canopy 190 or it can be of a material that gets darker and more opaque when an electrical voltage is applied to it, or it can get darker by other means, or it can simply be a piece of material with non-variable transparency properties.

D.) The Skeletal Structure 208, or the Mesh Wire Matting 200, or the Transparent Insulation 196 layers, or the Outside Covering 170, or any part or all of these can be replaced as helpful by a transparent or semi-transparent plastic or composite structure such as by a semi-transparent plastic composite structure (such as fiberglass solar structures). For example, a semi-transparent fiberglass structure that has two layers with an Air Gap 180 in between them can have the function of all the previously mentioned components of the Solar Canopy 190. The important thing is that two layers exist with an Air Gap 180 between them that can be purged to remove solar heating before it gets into the interior of the Solar Canopy 190.

E.) It is an option that the Solar Canopy 190 have no layer of Inner Insulation 178 or Air Gap 180 but just have the Canopy Blower 214 blowout heat from the Solar Canopy 190 as necessary.

F.) It is an option that during times of unusually high winds that the Air Gap Blower 210 and/or the Canopy Blower 214 are run at high enough pressure levels in order to put a slight pressure on the Air Gap 180 or Solar Canopy 190 to help resist unusually high wind loads on the Solar Canopy 190. Such a slight pressure can be the equivalent of the hydrostatic head of several inches of water or other pressures. It is also an option that the Solar Canopy 190 is not supported by structure but be supported partially or completely by the pressure of the Canopy Blower 214 or Air Gap Blower 210 or both.

G.) Any number of Canopy Blowers 214 or Air Gap Blowers 210 can be used where preferred or helpful. Fans can also be substituted for blowers. Natural convection in the Solar Canopy 190 or Air Gap 180 can be used where helpful.

H.) Anywhere on the layers of Transparent Insulation 196 can be mounted a layer of glass or plastic film or sheet which acts as a one-way mirror for infrared heat (or heat from other wavelengths), called the Infrared Reflector 222 (not shown in figures). This Infrared Reflector 222 allows infrared heat to pass through it to the interior of the Solar Canopy 190 after which the infrared heat is trapped inside the Solar Canopy 190 because the Infrared Reflector 222 reflects infrared heat back into the interior of the Solar Canopy 190. This function is especially useful on cold days when the Air Gap Blower 210 is off and it is desirable to collect as much solar heating as possible. If too much heat is taken into the Solar Canopy 190 then the blowers can be started as necessary. The Infrared Reflector 222 can be incorporated into any components/layers of the Solar Canopy 190 where helpful.

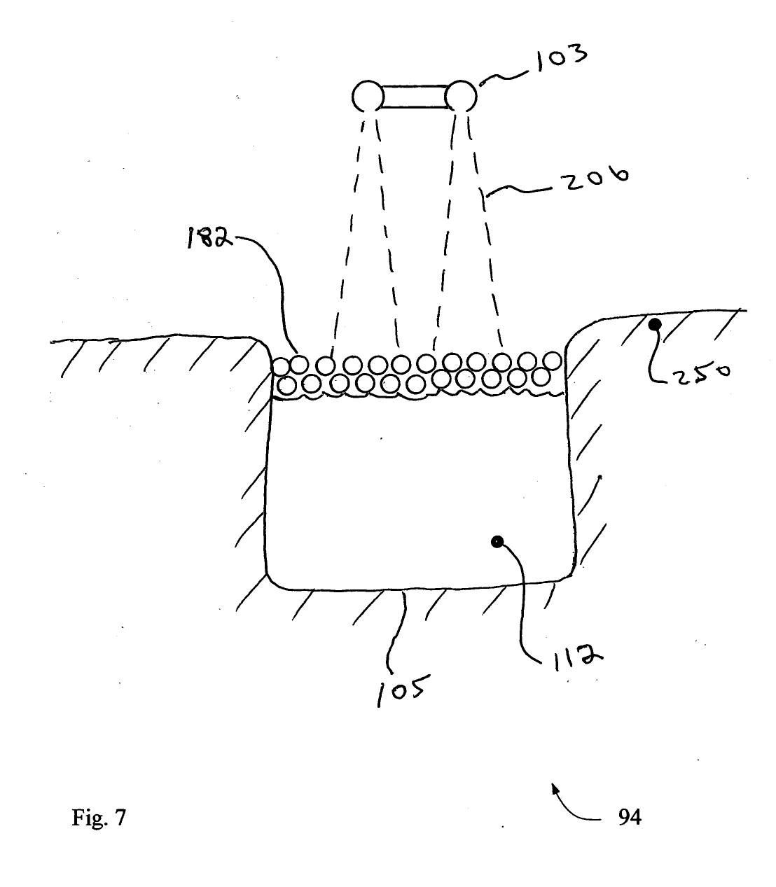

Waterfall Bioreactor Option: One option to the Falling Film Bioreactor 92 is a Waterfall Bioreactor 94 (FIG. 7). The Waterfall Bioreactor 94 is similar to the Falling Film Bioreactor 92 in that it is largely composed of a falling sheet of Culture Solution 112. However, with the Waterfall Bioreactor 94 the falling Culture Solution 112 is not supported by a Ramp 101 but simply falls from an Injection Manifold 103 or simply a High Point 280 to the Pool 105 as a Waterfall 206. The Injection Manifold 103 has a continuous slit in it or a series of holes or smaller slits or equivalent in order to form a falling Waterfall 206 when the Culture Solution 112 exits the Injection Manifold 103 or the Waterfall 206 can fall from an edge, or a High Point 280. The pressure moving the Culture Solution 112 to form the Waterfall 206 is a culture pump such as Culture Pump #1 or other means. The Waterfall Bioreactor 94 has many of the advantages of the Falling Film Bioreactor 92 while using less materials. These attributes and others will be discussed below:

Waterfall/Falling Film Width: A Waterfall Bioreactor 94 cross-sectional view is shown in FIG. 7. As with the Falling Film Bioreactor (FIG. 2) the width of the falling liquid sheet (i.e. the Waterfall 206) goes into the page of the figure. The width of the Waterfall Bioreactor 94 (or the Falling Film Bioreactor 92) can be any width the user prefers depending on the size of bioreactor required. Likewise each Pool 105 can have as many Waterfalls 206 (or Falling Film Ramps 101) as preferred or helpful. FIG. 7 shows a bioreactor with two such Waterfalls 206. Also, as with the Falling Film Bioreactor 92 the Waterfall Bioreactor 94 can have as many Injection Manifolds 103 as preferred or helpful or the Culture Solution 112 can simply fall over an edge. FIG. 7 shows two Injection Manifolds 103.

Floating Insulation: As seen in FIGS. 2 and 7 the top surface of the Culture Solution 112 in the Pool 105 is partially or totally covered with Floating Insulation 182. Floating Insulation 182 can be any kind of foam, bubble pack, or other material that has the preferred insulating properties and floats on or is above the surface of the Culture Solution 112 in the Pool 105 or surrounds the Culture Solution 112 to hellp control the Culture Solution's temperature. In the case of FIG. 7 the Floating Insulation 182 is comprised of two layers of ping-pong balls, although any number of layers can be used. The ping-pong balls insulate the top layer of the Pool 105 but are pushed out of the way of the Waterfall 206 but then re-cover the Pool 105 when the Waterfall 206 ceases (which may include, but not be limited to, night hours). Floating Insulation 182 can be of any size, shape, or material so long as the Waterfall 206 can flow past the floating objects and the floating objects cover the surface of the Pool 105 when and where preferred. It is also an option that Floating Insulation 182 be treated with or contain an anti-organism chemical such copper or other compounds so that the organisms' growth on the Floating Insulation 182 will be inhibited. Another option would be to use a Bioreactor 100 with no Floating Insulation 182. Any type of insulation can be added to a chemical production system utilizing this invention where helpful.

Waterfall Injection Angle: FIG. 7 shows the Waterfalls 206 as being injected (from the Injection Manifold 103) straight down to the Pool 105 parallel to the vertical axis. A Waterfall 206 can be injected at any angle relative to the Waterfall's vertical axis so long as the Waterfall 206 is intercepted by a Pool 105, pond, or tank, or other fluid receiving device

Fountain Bioreactor: Unlike the Waterfall 206 (FIG. 7) or the Falling Film 106 (FIG. 2) it is an option that the Culture Solutions 112 be injected in a relative upward direction (or some other angle to the vertical) as in a water fountain.

Falling Film, Waterfall, Fountain Bioreactor Height: A Falling Film, Waterfall, or Fountain Bioreactor 92, 94, 70 can be made of any height where helpful.

Collection of Organisms: As an option to using this invention in a continuous chemical production system, the organisms can simply be collected (i.e. for example: scooped up) from a bioreactor system and then use this invention to remove the Chemical Product 130 from the organisms. An example would be scooping up algae in the ocean and running it through an Oil Removal Tank(s) 120 and an Oil Settling Tank(s) 134 or other Chemical Product 130 removal mechanisms consistent with this invention.

Production of Chemical Product 130 On or External to the Organism's Body:

This invention can also be used with organisms that produce Chemical Product 130 on or external to their bodies, as opposed to forming Chemical Product 130 inside their cells or bodies. These are organisms that do not produce Chemical Product 130 within themselves but simply act as a catalyst or a component in the formation of Chemical Product 130 on or external to their bodies. The methods described in this disclosure is applicable to those organisms where waves, vibrations, or electrical voltage cause an increase in Chemical Product 130 production external to or attached to the outer surface of the organism's body or cause chemicals attached to the organisms' outer surface to separate from the organism.

Control Options: Any chemical production system using this method can be controlled through any means including no active control. Control systems can be active or passive, or electronic, or electric, or mechanical, or fluidic or others as the invention user prefers. The Liquid Level Equalization Line 164 shown in FIG. 1 is optional and keeps the liquid levels in the Pool 105 and Oil Settling Tank 134 level or near level with each other. As the liquid level in one of the two tanks rises above the liquid level of the other tank (or pool, vessel, or other container), the higher static pressure of the liquid in the tank with the higher liquid level will force open one of the two Check Valves 162 and flow liquid back to the tank with the lower liquid level, thus equalizing liquid levels in the two tanks. The two Check Valves 162 are arranged opposite to each other so one will allow liquid flow in one direction and the other Check Valve 162 will allow liquid flow in the opposite direction, thus keeping the liquid levels in both tanks at or near level with respect to each other. The cracking pressure on such Check Valves 162 would be within but not limited to a range of 0.1 to 1 psid. Such a Liquid Level Equalization Line 164 can be used with as many tanks as helpful. In addition, the tanks, pools, or ponds that are in systems that are utilizing this invention can be at any height relative to each other. FIG. 1 shows the pools/tanks as all level with each other, which is optional. If the tanks/pools are at different levels relative to each other, their liquid levels would have to be controlled or monitored as helpful to avoid undesirable overflow or drainage. Finally, the Controller/Power Supply 150 is a generic representation only. It can be a controller or a power supply or a combination of both or the two can be used as separate units or not used when helpful. Note that the Controller/Power Supply 150 of FIG. 4 is not shown in FIG. 1 for visual clarity. Any number of controllers or power supplies can be used.

Examples of Current Technology: This invention can be used in conjunction with components and equipment based on current technology when and where helpful, such as currently available Bioreactors 100. Examples of currently available bioreactors include tubular, plastic film, and non-photosynthesizing bioreactors and other types of bioreactors.

Falling Film Bioreactor Options: There are numerous options that apply to a Falling Film Bioreactor 92. Some of these options are:

The direction of the width of a Falling Film Bioreactor 92 is perpendicular to the page of FIG. 2 and can be of any size where helpful. Also, the Ramp 101 height, shape, quantity, number, orientation, angle, material, size, geometry, surface quality, configuration, material, or surface roughness can be of any type or value where helpful. In addition the Ramp(s) 101 can have steps in it or not. It is also an option to have angles (or structures of other shapes) going down the sides of the Ramps 101 to keep the Culture Solution 112 from spilling over the sides of the Ramps 101. Another option is that the Ramp(s) 101 can have a transparent or semi-transparent cover sheet over it such as glass, variable opacity sheet, plastic sheet, plastic film, Transparent Insulation 196, or other sheets or layers with a function of being a protective barrier to prevent water loss or contamination of the Culture Solution 112, or to help regulate temperature of the Culture Solution 112 (i.e. there is a space between the Ramp 101 and the cover sheet).

Overall System Options: Some, but not all, of the other options for this invention include:

The components of a Chemical Product 130 production system using this invention can be of any size, type, arrangement, geometry, orientation, or location where helpful so long as the function of this invention is achieved. Any material or process that has acceptable compatibility with the Growth Medium 132, Culture Solution 112, Chemical Product 130, and the other components can be used to make this invention. Components can be integrated with each other or used separately or used in series or in parallel with each other or any other helpful arrangement. Functions of components can also be combined with each other where helpful. Any component represented in the figures of this report can be used singularly or in a plurality. Any pumps in the system can be replaced with overflow or gravity fed methods where helpful or preferred.

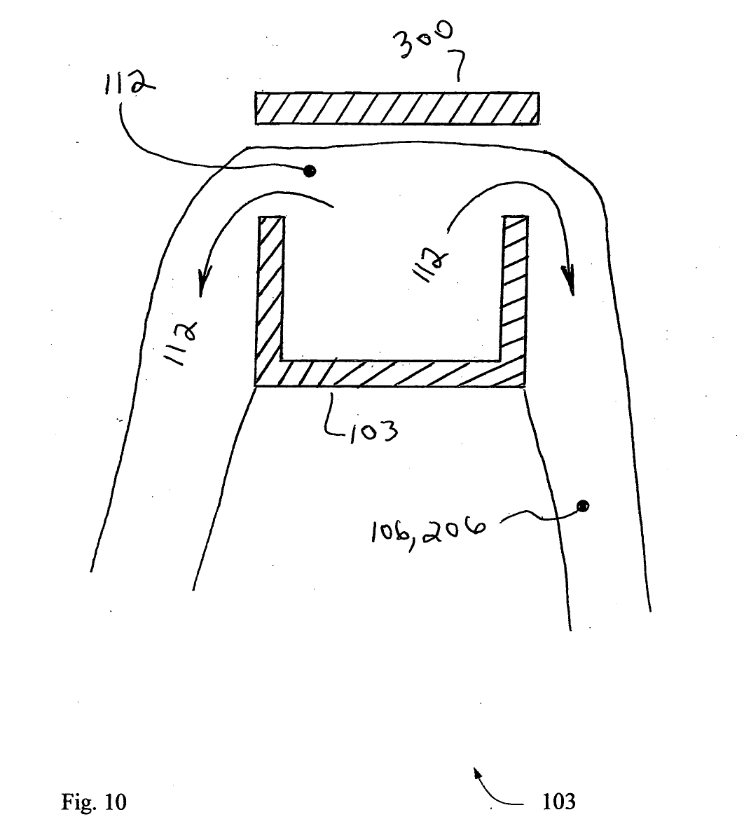

Injection Manifold Options: For the Falling Film Bioreactor 92 or the Waterfall Bioreactor 94 or the Injection Manifold 103 can be any plumbing device or plumbing configuration that distributes the Culture Solution 112 as helpful to achieve the purpose of this invention. The Injection Manifolds in FIGS. 2 and 7 are shown as round tubular manifolds that have the appropriate holes or slots for producing the Waterfall 206 or Falling Film 106. One of the options to this type of manifold would be a channel-shaped manifold of any shape whose top-side (nearest to the sky) is open (see FIG. 10). The channel manifold is blocked at the ends to contain the Culture Solution 112. An Injection Manifold 103 of the channel type would function by the Culture Solution 112 continuously overflowing the channel along the channel's length on one side or both sides and thus produce an even Waterfall 206 or Falling Film 106.

Wiring: Any Wiring 186 referred to in this disclosure or shown in the figures is for example only and is optional. Other wiring, power transmission, or control configurations are possible.

Number of Organisms: A chemical production system using this invention can incorporate or contain any number or combination of organisms or organism types or organism species as helpful to produce the Chemical Product 130.

Other Options and Variations: It is understood that other versions, variations, and options to the systems and components described in this method can be implemented and are within the scope of this disclosure.

Multiple Wave Regimes: In a chemical production system utilizing this invention where multiple Wave Generators 118 are used, it is an option that some of the individual Wave Generators 118 can be operated at wave regimes (i.e. vibration regimes) that differ from the wave regimes produced by other Wave Generators 118 in the system; thus producing a combination of wave regimes in the same system. It is also an option to operate individual Wave Generators 118 at different times with respect to other Wave Generators 118.

CO2 Absorption: One of the benefits of this invention is the absorption of carbon or carbon dioxide by the organisms through the synthesis of carbon containing compounds such as calcium carbonate, CaCO3 as a Chemical Product 130. By producing such compounds the organisms can perform the function of carbon sequestering.

Low Current Applications: This method is also valid in applications where the organisms are subjected to an electric voltage and parameters exist (such as a very low voltage) such that the current flowing through the organisms is so low as to be un-measurable. If applying the voltage increases separation of a Chemical Product 130 from an organism or increases the Chemical Product's 130 production rate, then the usage of the voltage is within the scope of this disclosure despite extremely low electric current measurement.

The Term ‘Organism’: In this disclosure the term ‘organism’ can refer to a single organism (such as a clump of seaweed or one algae cell) or a plurality of organisms (such as a cluster or colony of algae or other macro or micro-organisms).

Wave Generators: The Wave Generators 118 of this disclosure produce specified or helpful vibrations and are not to be confused with wave generators that produce simulated wind-induced water waves on beaches or shorelines for recreational surfers or for research into beach or shoreline erosion. The Wave Generators 118 of this disclosure are also not to be confused with wave generators that are electrical generators that produce electricity from the power of ocean waves.

Vegetable Oil as Biofuel: Vegetable Oil 116 can be used directly as a Biofuel in some applications or as an ingredient to Biofuels or other chemical products.

Ground: The term Ground 250 is a term for the earth as in ‘walking on the ground’. The Ground 250 may or may not be used as an electrical ground depending on how the local electrical equipment is hooked up. Also, some of the figures show components of this invention installed in or under Ground 250. This method of installation is entirely optional since components or entire systems can be located under the Ground 250, on the Ground 250, or above the Ground 250, or as other examples: floating in or on water, positioned underwater, or on concrete, or mounted or supported by in any other way that allows the function of this invention.

Resonance Chambers: Any tank, pool, pond, or vessel in which the organisms are stimulated, stressed, agitated, or vibrated by at least one Wave Generator 118 for the purposes of removing Chemical Product 130 from the organisms or for increasing the production rate of Chemical Product 130, such a tank, pool, pond, or other type of vessel, as an option, can be sized, designed, or produced to be a resonance chamber whereas the tank, pool, pond, or vessel has its resonance frequency equal to or approximately equal to at least one of the wave or vibration frequencies that are being generated and put out by the Wave Generator 118 or other Stimulus 290 such as an alternating current Voltage Potential 280.

FIG. 12, Air Drying System: FIG. 12 shows a system for recapturing water that is being blown out of the Solar Canopy 190 or out of a Bioreactor 100 as water vapor. As in FIG. 12 Water 320 containing Air 270 is being blown through the Solar Canopy 190 by a Canopy Blower 214 to help control the interior temperature of the Solar Canopy 190. The water containing Air 270 is blown to a Cryogenic Air Dryer 218. In the Cryogenic Air Dryer 218 a coolant fluid called the Cryogenic Coolant 330 is flowed through the Cryogenic Air Dryer 218 to condense Water 320 into a liquid form that is routed back to the Bioreactor's 100 vessel or Pool 105. The Bioreactor 100 shown in FIG. 12 is specifically a Falling Film Bioreactor 92. The Cryogenic Coolant 330 comes from a Cryo-Vessel 340 and is routed to the Cryogenic Air Dryer 218 through a Cryo-Feedline 350. The Water 320 travels from the Cryogenic Air Dryer back to the Pool 105 through a Water Return Line 310. The Cryogenic Air Dryer 218 is a heat exchanger that re-condenses water vapor back into liquid so the water is not lost from the chemical production system. The configurations of the Cryo-Vessel 340, the Cryo-Feedline 350, and the Water Return Line 310 as shown in FIG. 12 are only one configuration of many possible configurations. This system as shown in FIG. 12 can be configured in any way that gets Cryogenic Coolant 330 to the Cryogenic Air Dryer 218 and re-condensed Water 320 back to the Bioreactor 100, whatever type of bioreactor is used. As an example, the Water Return Line 310 can be routed on the Ground 250 instead of under the Ground 250 as shown in FIG. 12. Or the Water Return Line 310 can be routed above the Ground 250, or it can have any water moving device on it such as a pump. Or the Cryogenic Air Dryer 218 or any other system components can be located anywhere in the chemical production system such as in the Solar Canopy 190 or in a building so long as it can re-condense at least a portion of the evaporated Water 320 that is flowing out of the chemical production system.

In FIGS. 10 and 11 the Flow Deflector 300 is for redirecting flowing fluid. In FIG. 10 that fluid is Culture Solution 112 and in FIG. 11 the Fluid is Air 270. Flow Deflectors 300 are optional and can be used or not used as helpful.

Changes and modifications in the specifically described embodiments can be carried out without departing from the scope of the invention, which is intended to be limited by the scope of the claims.

### Method Details

Method:

A.) Organisms 50 are living in a Bioreactor 100 with a Culture Solution 112.

B.) The organisms 50 are synthesizing Chemical Product 130.

C.) The organisms 50 are exposed to at least one of the following Stimuli 290: Vibration Waves 184 or Voltage Potential 280.

D.) While the organisms 50 are exposed to the Stimulus 290 Chemical Product 130 is released from the organisms 50.

E.) The Chemical Product 130 is removed from the Culture Solution 112.

F.) The organisms 50 re-synthesize new Chemical Product 130.

G.) The sequence of organisms 50 living in a Bioreactor 100, organisms 50 synthesizing Chemical Product 130, Chemical Product 130 being removed from the organisms 50 by exposure to the Stimulus 290, Chemical Product 130 being removed from the Culture Solution 112, and then the organisms 50 re-synthesize new Chemical Product 130 is repeated at least once.

Method:

A.) Organisms 50 are living in a Bioreactor 100 with a Culture Solution 112.

B.) The organisms 50 are exposed to at least one of the following Stimuli 290: Vibration Waves 184 or Voltage Potential 280;

C.) While exposed to the Stimulus 290 the organisms 50 synthesize Chemical Product 130 at a higher rate than organisms 50 not exposed to the Stimulus 290.

I claim:

1. A Chemical Product Production System where: organisms that are micro-organisms are cultured in a Bioreactor with a Culture Solution to produce at least one Chemical Product within the micro-organisms' cellular bodies, and whereas at least one Wave Generator subjects the micro-organisms to Vibration Waves to cause the removal of Chemical Product from the cellular bodies of the micro-organisms without destroying at least a portion of the micro-organisms, and whereas the micro-organisms are continuously processed for Chemical Product.

2. The Chemical Product Production System of claim 1 whereas a Chemical Product is removed from the Culture Solution by a Coalescing Pack treated with Bio-Repellant material.

3. The Chemical Product Production System of claim 1 whereas micro-organisms are separated from a Chemical Product by a BioFilter treated with a Bio-Repellant material.

4. The Chemical Product Production System of claim 1 whereas the organisms are macro-organisms.

5. The Chemical Product Production System of claim 1 whereas the Stimulus applied to the Organisms is a Voltage Potential.

6. The Chemical Product Production System of claim 1 whereas organisms are subjected to both Vibration Waves and electrical voltage.

7. The Chemical Product Production System of claim 1 whereas a Chemical Product is produced by a batch process.

8. The Chemical Product Production System of claim 1 whereas a Chemical Product is produced by a semi-continuous process.

9. The Chemical Product Production System of claim 1 where at least a portion of the Bioreactor is a Falling Film Bioreactor with a Culture Solution flowing down a surface, called a Ramp, and with the Culture Solution continuously being re-circulated back to the Ramp to flow down the Ramp continuously.

10. The Chemical Product Production System of claim 1 where at least a portion of the Bioreactor is a Waterfall Bioreactor with a Culture Solution falling through Air from a High Point as a Waterfall of Culture Solution that is continuously being re-circulated back to a High Point to continuously fall through Air.

11. The Chemical Product Production System of claim 1 where at least a portion of the Bioreactor is a Fountain Bioreactor with a Culture Solution propelled upward through air as a spray of Culture Solution that falls through Air and is continuously being re-circulated upward through Air as a spray.

12. The Chemical Product Production System of claim 1 where at least a portion of the Bioreactor is a Landfill Bioreactor.

13. The Chemical Product Production System of claim 1 where at least a portion of a Chemical Product Production System is contained within a Solar Canopy that is comprised of at least two layers of at least partially transparent Transparent Insulation with an Air Gap in between the layers of Transparent Insulation, and whereas warm air collecting between the layers of Transparent Insulation is continuously purged out of the Air Gap to help control the temperature of the Culture Solution and the interior temperature of the Solar Canopy.

14. The Chemical Product Production System of claim 1 whereas organisms produce a Chemical Product and whereas Vibration Waves applied to the organisms increases the rate of Chemical Product production.

15. The Chemical Product Production System of claim 1 whereas organisms produce a Chemical Product and whereas a voltage potential applied to the organisms increases the rate of Chemical Product production.

16. The Chemical Product Production System of claim 1 whereas a vessel where the Organisms are exposed to a vibration Stimulus, the said vessel is sized such as to have a Resonance Frequency that is at least approximately equal to at least one of the frequencies generated by the vibration Stimuli.

17. The Chemical Product Production System of claim 1 whereas the Chemical Product is separated from the Culture Solution with bubbles gas known as Air Bubbles.

18. A method where: Organisms are living in a Bioreactor with a Culture Solution; and whereas the organisms are synthesizing Chemical Product; and whereas the organisms are exposed to at least one of the following Stimuli: Vibration Waves or Voltage Potential; and whereas while the organisms are exposed to the Stimulus Chemical Product is released from the organisms; and whereas the Chemical Product is removed from the Culture Solution; and whereas the organisms re-synthesize new Chemical Product; and whereas the sequence of organisms living in a Bioreactor, organisms synthesizing Chemical Product, Chemical Product being removed from the organisms by exposure to the Stimulus, Chemical Product being removed from the Culture Solution, and then the organisms re-synthesize new Chemical Product is repeated at least once.

19. A method where: Organisms are living in a Bioreactor with a Culture Solution; and whereas the organisms are exposed to at least one of the following Stimuli: Vibration Waves or Voltage Potential; and whereas while exposed to the Stimulus the organisms synthesize Chemical Product at a higher rate than organisms not exposed to the Stimulus.

20. The Chemical Product Production System of claim 1 whereas evaporated Water is condensed in a Cryogenic Air Dryer and routed to the Chemical Product Production System.
